(19) United States
(12) Patent Application Publication
(10) Pub. No.: US 2024/0025904 A1
(43) Pub. Date: Jan. 25, 2024

(54) PDE9 INHIBITORS FOR TREATING CARDIAC FAILURE

(71) Applicants: Cardurion Pharmaceuticals, Inc., Burlington, MA (US); Vanderbilt University, Nashville, TN (US)

(72) Inventors: Rahul Dilip BALLAL, Boston, MA (US); Thiago Trovati MACIEL, Boston, MA (US); Deepak GUPTA, Nashville, TN (US); Thomas WANG, Nashville, TN (US)

(21) Appl. No.: 18/033,662

(22) PCT Filed: Oct. 26, 2021
(86) PCT No.: PCT/US2021/056696

§ 371 Date: Apr. 25, 2023

Related U.S. Application Data
(60) Provisional application No. 63/106,301, filed on Oct. 27, 2020.

(51) Int. Cl. C07D 487/04; A61K 38/08; A61P 9/04; A61P 3/04

(52) U.S. Cl. CPC C07D 487/04; A61K 38/085; A61P 9/04; A61P 3/04

(57) ABSTRACT

The present disclosure relates to PDE9 inhibitors, compositions comprising the PDE9 inhibitors, and methods of using the PDE9 inhibitors and compositions for treatment of cardiac failure.

### CROSS REFERENCE

This application claims the benefit of U.S. Application No. 63/106,301, filed Oct. 27, 2020, which is hereby incorporated by reference in its entirety.

### BACKGROUND OF THE INVENTION

Heart failure (HF) or cardiac failure, is when the heart is unable to pump sufficiently to maintain blood flow to the body. Common causes of heart failure include among others coronary artery disease, high blood pressure, atrial fibrillation, valvular heart disease, excess alcohol use, infection, and cardiomyopathy. Heart failure is a common, costly, and potentially fatal condition. In 2015, it affected about 40 million people globally. Overall 2% of adults have heart failure and in those over the age of 65, this increases to 6-10%. The risk of death is about 35% the first year after diagnosis. There is an urgent need to develop improved therapies for cardiac failure and other associated cardiac diseases.

### SUMMARY OF THE DISCLOSURE

The present disclosure provides methods using Compound 1 and/or a pharmaceutical composition comprising Compound 1 or a pharmaceutically acceptable salt, solvate, or polymorph thereof, to treat cardiac disease, including cardiac failure, cardiac fibrosis, and myocardial inflammation.

An aspect of the present disclosure comprises a method of treating cardiac failure in a patient in need thereof, comprising administering a PDE9 inhibitor, for example 6-[4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof to a subject in need thereof, wherein the compound is administered at a dose of more than or less than 10 mg/kg per patient weight.

In some embodiments, the cardiac failure is acute, chronic, or congestive cardiac failure. In some embodiments, wherein the cardiac failure is diabetes induced, autoimmune based, or inflammatory based cardiac failure. In some embodiments, the cardiac failure is cardiac failure with a preserved ejection fraction or with a reduced ejection fraction.

An aspect of the present disclosure comprises a method of treating cardiac fibrosis in a patient in need thereof, comprising administering a PDE9 inhibitor, for example 6-[4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof to a subject in need thereof.

In some embodiments, the treating of cardiac fibrosis further comprise decreasing accumulation of fibronectin and/or collagen type I and II.

An aspect of the present disclosure comprises a method of treating myocardial inflammation in a patient in need thereof, comprising administering a PDE9 inhibitor, for example 6-[4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof to a subject in need thereof.

An aspect of the present disclosure comprises a method of decreasing ANP and/or BNP in a patient in need thereof, comprising administering a PDE9 inhibitor, for example 6-[4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt, solvate, or polymorph thereof to a subject in need thereof.

In some embodiments, the ANP is decrease by about 5%, 10%, 20%, 30%, 40%, or 50%, or more compared to pretreatment levels. In some embodiments, the BNP is decrease by about 5%, 10%, 20%, 30%, 40%, or 50%, or more compared to pretreatment levels.

In some embodiments of any of the methods, the PDE9 inhibitor is administered to the patient at a dose of between about 1 mg/kg to about 10 mg/kg per body weight. In some embodiments, the PDE9 inhibitor is administered to a patient at a dose of about 2 mg/kg, about 3 mg/kg, about 4 mg/kg, about 5 mg/kg, about 6 mg/kg, about 7 mg/kg, about 8 mg/kg, or about 9 mg/kg per body weight. In some embodiments of any of the methods, the PDE9 inhibitor is administered to the patient at a dose of between about 10 mg/kg to about 500 mg/kg per body weight. In some embodiments, the PDE9 inhibitor is administered to a patient at a dose of about 50 mg/kg, about 100 mg/kg, about 150 mg/kg, about 200 mg/kg, or about 250 mg/kg per body weight. In some embodiments, the PDE9 inhibitor is administered to a patient at a dose of about 60 mg/kg or about 100 mg/kg per body weight. In some embodiments, the PDE9 inhibitor is administered to the patient at at about 100 mg to about 800 mg per dose. In some embodiments, the PDE9 inhibitor is administered to the patient at about 100 mg, 200 mg, 300 mg, 400 mg, 500 mg, 600 mg, 700 mg or 800 mg per dose. In some embodiments, the PDE9 inhibitor is administered QD, BID, or TID.

In some embodiments, the PDE9 inhibitor is administered with at least one additional therapeutic agent. In some embodiments, the additional therapeutic agent is selected from an angiotensin transferase inhibitor (ACEI), a β-receptor blocker, a mineralocorticoid/aldosterone receptor antagonist (MRA), a diuretic, an angiotensin receptor neprilysin inhibitor (ARNI), a neprilysin inhibitor (NEPI), an angiotensin II receptor blocker (ARB), a vasodilator, and a hydralazine (HYD) or isosorbide dinitrate (SND), or a combination thereof. In some embodiments, the addtional therapeutic agent is selected from hydroxy urea (HU), captopril, enalapril, lisinopril, trandolapril, bisoprolol, carvedilol, metoprolol succinate, nebivolol, eplerenone, spirolactone, sacubitril, ivabradine, candesartan, valsartan, digoxin, deslanoside, dopamine, dobutamine, dopexamine, milrinone, enoximone, phosphocreatine, cyclohexylethylamine, nitroglycerin, isosorbide dinitrate, sodium nitroprusside, prazosin, ivabradine, candesartan, valsartan, furosemide, bumetanide, torasemide, bendrofluazide, hydrochlorothiazide, metolazone, indapamide, amiloride, and triamterene. In some embodiments, the PDE9 inhibitor and the a least one additional therapeutic are administed concurrently or sequentially. In some embodiments, the PDE9 inhibitor is administerd orally. In some embodiments, the PDE9 inhibitor is administerd daily. In some embodiments, the PDE9 inhibitor is administerd for between 1 to 7 days. In some embodiments, the PDE9 inhibitor is administed for at least 7 days.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A and 1B show that Compound 1 (IMR-687) decreases heart size and cardiomyocyte hypertrophy in the Angiotensin II infusion model for 6 weeks of dosing at 60 and 100 mg/kg.

FIGS. 2A and 2B shows that Compound 1 (IMR-687) decreases heart size and cardiomyocyte hypertrophy in the nephrectomy-aldosterone model after 4 weeks of dosing at 60 and 100 mg/kg.

FIGS. 3A and 3B shows that Compound 1 (IMR-687), in combination with Angiotensin II (left side) or nephrectomy-aldosterone (right side), decreases ANP and BNP markers of cardiac dysfunction after dosing with 60 and 100 mg/kg. FIG. 3A shows ANP biomarkers while FIG. 3B shows BNP biomarkers.

FIGS. 4A and 4B show that HFpEF, PDE5 and PDE9 expression is increased after administration of Compound 1 (IMR-687), in combination with Angiotensin II (4A) or nephrectomy-aldosterone (4B), leading to decreased cGMP levels, lower PKG activity, and excessive Ca⁺⁺ channel activity.

FIG. 5 shows that angiotensin II induces myocardial fibrosis through expression of TGF-ß, accumulation of fibronectin and collagen type I and II (Scientific Reports|6:37635|DOI: 10.1038/srep37635).

FIGS. 6A and 6B shows that Compound 1 (IMR-687), in combination with Angiotensin II, decreases heart fibrosis by blocking TGF-ß1 and downstream targets (Fibronectin and Collagen type I and III). FIG. 6B shows periodic acid-Schiff stain (extracellular matrix rich in glycogen, and mucosubstances such as glycoproteins, glycolipids and mucins.

FIG. 7 shows that Compound 1 (IMR-687), in combination with nephrectomy-aldosterone, decreases heart fibrosis by blocking TGF-ß1 and downstream targets (Fibronectin and Collagen type I and III).

FIG. 8A-8C shows that Compound 1 (IMR-687), in combination with Angiotensin II (8A) or nephrectomy-aldosterone (8B) decreases markers of myocardial inflammation. C Kelly R A et al. Circulation. 1997; 95:778-781.

FIG. 9 shows that PDE9 is overexpressed in reticulocytes and neutrophils in sickle cell disease as well as in the myocardium of patients with heart failure with preserved ejection fraction, suggesting despite elevated natriuretic peptide levels in these conditions, cGMP may be relatively depleted.

FIG. 10 shows baseline characteristics of subjects randomized to Compound 1+HU or HU alone. The subjects were normotensive with both systolic and diastolic blood pressure within normal range.

FIG. 11 shows mean NT-proBNP levels. In the Compound 1+HU group, the mean baseline and 4-month follow-up NT-proBNP levels were 467 and 340 pg/ml, respectively (mean decrease of 127 pg/ml or 27% reduction). In the HU group, the mean baseline and 4-month follow-up NT-proBNP levels were 343 and 436 pg/ml, respectively (mean increase of 93 pg/ml or 27.% higher). A greater than 50% reduction in NT-proBNP levels was seen at 4-months in 30% of Compound 1+HU treated subjects, but none of the HU alone treated subjects.

FIG. 12 shows the change in NT-proBNP at 4 months as a function of baseline NT-proBNP according to randomization is shown on this figure. In an adjusted model for the 4 months change in NT-proBNP, it was found that the main effect of Compound 1+HU was significant (p=0.01), but the interaction effect of Compound 1+HU by baseline NT-proBNP level was highly significant (p<0.0001). In subjects with baseline NT-proBNP values ≥400 pg/ml, Compound 1+HU was associated with an average approximately 68% reduction in NT-proBNP between baseline and 4 months compared with an average 28% increase with HU alone. In contrast, among subjects with baseline NT-proBNP levels <400 pg/ml, 4-month treatment with Compound 1+HU did not significantly change NT-proBNP levels compared with HU alone. Compound 1+HU was not associated with changes in heart rate or blood pressure over 4 months compared with HU alone, suggesting these findings are not due to hemodynamic factors.

### DETAILED DESCRIPTION OF THE INVENTION

Phosphodiesterases (PDEs) are a family of enzymes degrading cyclic nucleotides and thereby regulating the cellular levels of second messengers throughout the entire body. PDEs represent attractive drug targets, as proven by a number of compounds that have been introduced to clinical testing and the market, respectively. PDEs are encoded by 21 genes that are functionally separated into 11 families differing with respect to kinetic properties, substrate selectivity, expression, localization pattern, activation, regulation factors and inhibitor sensitivity. The function of PDEs is the degradation of the cyclic nucleotide monophosphates cyclic Adenosine Monophosphate (cAMP) and/or Guanosine Monophosphate (cGMP), which are important intracellular mediators involved in numerous vital processes including the control of neurotransmission and smooth muscle contraction and relaxation.

PDE9 is cGMP specific (Km cAMP is >1000× for cGMP) and is hypothesized to be a key player in regulating cGMP levels as it has the lowest Km among the PDEs for this nucleotide. PDE9 is expressed throughout the brain at low levels with the potential for regulating basal cGMP.

In the periphery, PDE9 expression is highest in prostate, intestine, kidney and hematopoietic cells, enabling therapeutic potential in various non-CNS indications.

In the present disclosure, a PDE9 inhibitor (for example, Compound 1) is used for treatment for cardiac diseases, including but not limited to cardiac failure, cardiac dysfunction and cardiomyopathy.

### I. Compounds

In the context of the present disclosure a compound is considered to be a PDE9 inhibitor if the amount required to reach the 50% inhibition level of any of the three PDE9 isoforms is 10 micromolar or less, preferably less than 9 micromolar, such as 8 micromolar or less, such as 7 micromolar or less, such as 6 micromolar or less, such as 5 micromolar or less, such as 4 micromolar or less, such as 3 micromolar or less, more preferably 2 micromolar or less, such as 1 micromolar or less, in particular 500 nM or less. In preferred embodiments the required amount of PDE9 inhibitor required to reach the IC₅₀ level of PDE9 is 400 nM or less, such as 300 nM or less, 200 nM or less, 100 nM or less, or even 80 nM or less, such as 50 nM or less, for example 25 nM or less.

In some embodiments, the PDE9 inhibitor of the present disclosure has low or no blood brain barrier penetration. For example, the ratio of the concentration of a PDE9 inhibitor of the present disclosure in the brain to the concentration of it in the plasma (brain/plasma ratio) may be less than about 0.50, about 0.40, about 0.30, about 0.20, about 0.10, about 0.05, about 0.04, about 0.03, about 0.02, or about 0.01. The brain/plasma ratio may be measured 30 min or 120 min after administration of the PDE9 inhibitor.

In some embodiments, the PDE9 inhibitor may be any imidazo pyrazinone PDE9 inhibitor disclosed in WO 2013/053690, the contents of which is incorporated herein by reference in its entirety.

Where compounds of the present invention contain one or more chiral centers reference to any of the compounds will, unless otherwise specified, cover the enantiomerically or diastereomerically pure compound as well as mixtures of the enantiomers or diastereomers in any ratio.

In an embodiment, the PDE9 inhibitor is a compound having the structure of Formula (I), or a pharmaceutically acceptable salt, solvate, or polymorphic thereof:

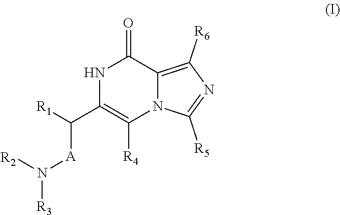

wherein R² is cyclized with either R¹ or R³;
wherein R¹, R², and R³ are
   R¹, when cyclized with R² is

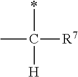

wherein R⁷ is selected from the group consisting of H, —CH₃, —C₂H₅, and C₃H₇;
wherein * denotes the cyclization point; and
R¹, when not cyclized, is selected from the group consisting of H and

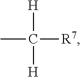

wherein R⁷ is selected from the group consisting of H, —CH₃, —C₂H₅, and C₃H₇;
R² is a compound selected from the group consisting of

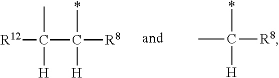

wherein R⁸ and R¹² independently are selected from the group consisting of H, —CH₃, —C₂H₆, and —C₃H₇,
wherein * denotes the cyclization point; and
R³, when cyclized with R² is

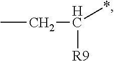

wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R³, when not cyclized, is

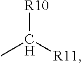

wherein
   R¹⁰ is selected from the group consisting of H, —CH₃, and —C₂H₅; and
   R¹¹ is selected from the group consisting of C₆-C₁₉ aryl, substituted C₆-C₁₀ aryl, C₃-C₉ heteroaryl, substituted C₃-C₉ heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R⁶ is selected from the group consisting of hydrogen, F, Cl, CN, —CH₃, —C₂H₅, —C₃H₇, and —CF₃; and
A is absent or —CH₂.

In some embodiments, the PDE9 inhibitor having the structure of Formula (I) is selected from the group consisting of: 3-(4-fluorophenyl)-6-((3-(pyridin-4-yloxy)azetidin-1-yl)methyl)imidazo[1,5-a]pyrazin-8(7H)-one (Compound P1), 6-[3-(pyridin-3-yloxy)-azetidin-1-ylmethyl]-3-(tetrahydro-pyran-4-yl)-7H-imidazo[1,5-a]pyrazin-8-one (Compound P2), 6-((3S,4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1, or Compound 1), and 6-((3R,4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2).

In some embodiments, the PDE9 inhibitor is 6-((3S,4S)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 1, Compound 1).

In some embodiments, the PDE9 inhibitor is 6-((3R,4R)-4-methyl-1-pyrimidin-2-ylmethyl-pyrrolidin-3-yl)-3-(tetrahydro-pyran-4-yl)-7H-imidazo[1,5-a]pyrazin-8-one (P3, enantiomer 2).

In some embodiments, the PDE9 inhibitor is selected from the group consisting of:

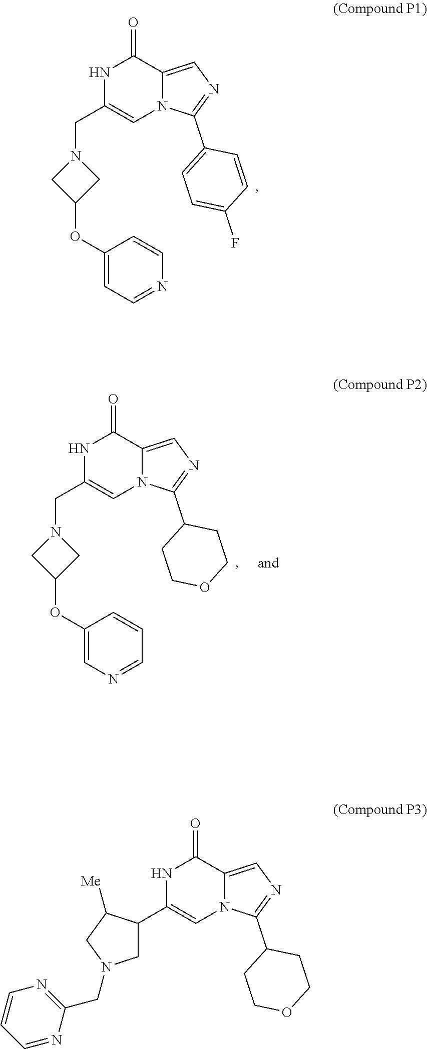 [SMILES: O=c1[nH]c(CN2CC(Oc3ccncc3)C2)cn2c(-c3ccc(F)cc3)ncc12 ; O=c1[nH]c(CN2CC(Oc3cccnc3)C2)cn2c(C3CCOCC3)ncc12 ; CC1CN(Cc2ncccn2)CC1c1cn2c(C3CCOCC3)ncc2c(=O)[nH]1]

in racemic form or in enantiomerically enriched or pure form; or a pharmaceutically acceptable salt, solvate, or polymorph thereof.

In some embodiments, the PDE9 inhibitor is Compound 1 or a pharmaceutically acceptable salt, solvate, or polymorph thereof. A racemate form of Compound 1 (otherwise defined as Compound P3) and an anhydrous form of Compound 1 have been described in WO 2013/053690 and WO 2017/005786. Crystalline forms have been described in WO 2019/226944. Compound 1 (IMR-687) has the following structure:

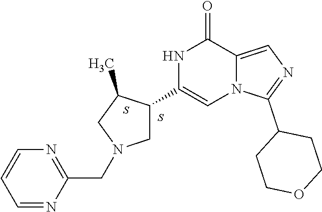

6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (compound P3 enantiomer 1 also known as compound P3.1); Formula C₂₁H₂₆N₆O₂; calculated molecular weight about 394 g/mol.

### II. Pharmaceutical Composition

The present disclosure further provides for a method of treating heart disease by administering the patient in need thereof a pharmaceutical composition comprising a therapeutically effective amount of any of the PDE9 inhibitors and a pharmaceutically acceptable carrier or diluent. In some embodiments, the pharmaceutical composition comprises a therapeutically effective amount of a compound having the structure of Formula (I), a pharmaceutically acceptable salt, solvate, or polymorph thereof, and a pharmaceutically acceptable carrier or diluent or excipient. In some embodiments, the pharmaceutical composition comprises a therapeutically acceptable amount of Compound 1 or a pharmaceutically acceptable salt, solvate, or polymorph thereof, and a pharmaceutically acceptable carrier or diluent or excipient.

### Pharmaceutically Acceptable Salts

The present disclosure also comprises salts of the PDE9 inhibitors, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in Berge, S. M. et al., J. Pharm. Sci. 1977, 66, 2, the contents of which are hereby incorporated by reference.

Furthermore, the compounds of this disclosure may exist in unsolvated as well as in solvated forms with pharmaceutically acceptable solvents such as water, ethanol and the like. In general, the solvated forms are considered equivalent to the unsolvated forms for the purposes of this disclosure.

In some embodiments, the pharmaceutical composition comprises Compound 1 as the solvated, unsolvated, or crystalline/polymorph form. In some embodiments, Compound 1 is present as the unsolvated form. In some embodiments, Compound 1 is present as the solvated form. In some embodiments, Compound 1 is present as the crystalline form. In some embodiments, Compound 1 is present as the monohydrate crystalline form.

### Formulations

The compounds of the disclosure may be administered alone or in combination with pharmaceutically acceptable carriers, diluents or excipients, in either single or multiple doses.

The pharmaceutical compositions according to the disclosure may be formulated with pharmaceutically acceptable carriers or diluents as well as any other known adjuvants and excipients in accordance with conventional techniques such as those disclosed in Remington: The Science and Practice of Pharmacy, 22nd Edition, Gennaro, Ed., Mack Publishing Co., Easton, PA, 2013.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general health and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use. Other suitable administration forms include, but are not limited to, suppositories, sprays, ointments, creams, gels, inhalants, dermal patches and implants.

For parenteral routes such as intravenous, intrathecal, intramuscular and similar administration, typical doses are on the order of half the dose employed for oral administration.

The present disclosure also provides a process for making a pharmaceutical composition comprising admixing a therapeutically effective amount of a compound of the present disclosure and at least one pharmaceutically acceptable carrier or diluent.

The compounds of this disclosure are generally utilized as the free substance or as a pharmaceutically acceptable salt thereof. Such salts are prepared in a conventional manner by treating a solution or suspension of a compound of the present disclosure with a molar equivalent of a pharmaceutically acceptable acid. Representative examples of suitable organic and inorganic acids are described above.

For parenteral administration, solutions of the compounds of the present disclosure in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of the present disclosure may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of the present disclosure and a pharmaceutically acceptable carrier are then readily administered in a variety of dosage forms suitable for the disclosed routes of administration. The formulations may conveniently be presented in unit dosage form by methods known in the art of pharmacy.

Formulations of the present disclosure suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit. If a liquid carrier is used, the preparation may be in the form of a syrup, emulsion, soft gelatin capsule or sterile injectable liquid such as an aqueous or non-aqueous liquid suspension or solution.

The pharmaceutical compositions of the disclosure may be prepared by conventional methods in the art. For example, tablets may be prepared by mixing the active ingredient with ordinary adjuvants and/or diluents and subsequently compressing the mixture in a conventional tabletting machine prepare tablets. Examples of adjuvants or diluents comprise: corn starch, potato starch, talcum, magnesium stearate, gelatin, lactose, gums, and the like. Any other adjuvants or additives usually used for such purposes such as colorings, flavorings, preservatives etc. may be used provided that they are compatible with the active ingredients.

The pharmaceutical compositions may comprise at least 10%, 20%, 30%, 40%, 50%, 60%, 70%, 80%, or 90% by weight PDE9 inhibitor (e.g. Compound 1), or a pharmaceutically acceptable salt, solvate, or polymorph thereof. In some embodiments, the pharmaceutical composition may comprise at least 90%, 91%, 92%, 93%, 94%, 95%, 96%, 97%, 98%, or 99% by weight of Compound 1, or a pharmaceutically acceptable salt, solvate, or polymorph thereof.

In some embodiments, Compound 1 or a pharmaceutically acceptable salt, solvate, or polymorph thereof is formulated as a composition for oral administration. For example, it may be in a solid tablet form. The composition for oral administration comprises at least a filler and/or a processing aid. The processing aid may be a glidant or a lubricant. The composition for oral administration may also comprise a coating. In some embodiments, the composition for oral administration comprises microcrystalline cellulose and/or pregelatinized starch as fillers.

In some embodiments, the composition for oral administration comprises colloidal silicon dioxide and/or magnesium stearate as processing aids. In some embodiments, the composition for oral administration comprises Opadry® II white film coating. Opadry® II is a high productivity, water soluble, pH independent complete dry powder film coating system containing polymer, plasticizer and pigment which allows for immediate disintegration for fast, active release. In some embodiments, the composition for oral administration comprises purified water, which is removed during processing.

In some embodiments, the tablet comprises a coating between about 5% to about 20% (e.g., about 5%, 10%, 15% or 20%) by weight of the total weight of the tablet.

In the embodiment, the tablet comprises pregelatinized starch between about 4% to about 6% by weight of the total weight of the tablet.

In the embodiment, the tablet comprises colloidal silicon dioxide between about 1% to about 2.5% by weight of the total weight of the tablet.

In the embodiment, the tablet comprises magnesium stearate between about 0.5% to about 1.5% by weight of the total weight of the tablet.

In some embodiments, the tablet comprises pregelatinized starch, colloidal silicon dioxide, and magnesium stearate at a weight ratio of 5:2:1.

In some embodiments, the tablet comprises a coating of around 10% by weight of the tablet.

In some embodiments, the composition comprising Compound 1, or a pharmaceutically acceptable salt, solvate, or polymorph thereof is stored at controlled room temperature (20-25° C.).

In some embodiments, the composition comprising Compound 1, or a pharmaceutically acceptable salt, solvate, or polymorph thereof is protected from light.

In some embodiments, the composition comprising Compound 1, or a pharmaceutically acceptable salt, solvate, or polymorph thereof is taken with food.

### Dosing

Typical oral dosages range from about 0.001 to about 100 mg/kg body weight per day, or any range therein. Typical oral dosages also range from about 0.01 to about 50 mg/kg body weight per day, or any range therein. Typical oral dosages further range from about 0.05 to about 10 mg/kg body weight per day, or any range therein. Oral dosages are usually administered in one or more dosages, typically, one to three dosages per day. The exact dosage will depend upon the frequency and mode of administration, the gender, age, weight and general health of the subject treated, the nature and severity of the condition treated and any concomitant diseases to be treated and other factors evident to those skilled in the art.

In some embodiments, the PDE9 inhibitor (e.g. Compound 1), or a pharmaceutically acceptable salt, solvate, or polymorph thereof is administered to a patient in need thereof at a dosing of less than 6.0 mg/kg or less than about 4.0 mg/kg. For example, Compound 1, or a pharmaceutically acceptable salt, solvate, or polymorph thereof is administered at a dosing of between about 0.3 to about 3.0 mg/kg, or about 0.3 to about 1.0 mg/kg, or any range therein. The patient may have a cardiac dysfunction. The patient may be an adult (≥18 years old) or a child (<18 years old).

In some embodiments, the patient receives Compound 1, or a pharmaceutically acceptable salt, solvate, or polymorph thereof at about 1 mg/kg. In some embodiments, the patient receives Compound 1, or a pharmaceutically acceptable salt, solvate, or polymorph thereof at about 3 mg/kg. In some embodiments, the patient receives Compound 1, or a pharmaceutically acceptable salt, solvate, or polymorph thereof at about 6 mg/kg. In some embodiments, the patient receives Compound 1, or a pharmaceutically acceptable salt, solvate, or polymorph thereof at about 8.0 mg/kg. In some embodiments, the patient receives Compound 1, or a pharmaceutically acceptable salt, solvate, or polymorph thereof at about 10 mg/kg. In some embodiments, the patient receives Compound 1, or a pharmaceutically acceptable salt, solvate, or polymorph thereof at about 20 mg/kg. In some embodiments, the patient receives Compound 1, or a pharmaceutically acceptable salt, solvate, or polymorph thereof at about 50 mg/kg. In some embodiments, the patient receives Compound 1, or a pharmaceutically acceptable salt, solvate, or polymorph thereof at about 60 mg/kg. In some embodiments, the patient receives Compound 1, or a pharmaceutically acceptable salt, solvate, or polymorph thereof at about 100 mg/kg. In some embodiments, the patient receives Compound 1, or a pharmaceutically acceptable salt, solvate, or polymorph thereof at about 200 mg/kg.

In some embodiments, the patient receives at least about 1 mg/kg of Compound 1, or a pharmaceutically acceptable salt, solvate, or polymorph thereof per the weight of the patient. In some embodiments, the patient receives at least about 2 mg/kg of Compound 1, or a pharmaceutically acceptable salt, solvate, or polymorph thereof per the weight of the patient. In some embodiments, the patient receives at least about 3 mg/kg of Compound 1, or a pharmaceutically acceptable salt, solvate, or polymorph thereof per the weight of the patient. In some embodiments, the patient receives at least about 4 mg/kg of Compound 1, or a pharmaceutically acceptable salt, solvate, or polymorph thereof per the weight of the patient. In some embodiments, the patient receives at least about 5 mg/kg of Compound 1, or a pharmaceutically acceptable salt, solvate, or polymorph thereof per the weight of the patient. In some embodiments, the patient receives at least about 6 mg/kg of Compound 1, or a pharmaceutically acceptable salt, solvate, or polymorph thereof per the weight of the patient. In some embodiments, the patient receives at least about 7 mg/kg of Compound 1, or a pharmaceutically acceptable salt, solvate, or polymorph thereof per the weight of the patient. In some embodiments, the patient receives at least about 8 mg/kg of Compound 1, or a pharmaceutically acceptable salt, solvate, or polymorph thereof per the weight of the patient. In some embodiments, the patient receives at least about 9 mg/kg of Compound 1, or a pharmaceutically acceptable salt, solvate, or polymorph thereof per the weight of the patient. In some embodiments, the patient receives at least about 10 mg/kg of Compound 1, or a pharmaceutically acceptable salt, solvate, or polymorph thereof per the weight of the patient. In some embodiments, the patient receives at least about 20 mg/kg of Compound 1, or a pharmaceutically acceptable salt, solvate, or polymorph thereof per the weight of the patient. In some embodiments, the patient receives at least about 30 mg/kg of Compound 1, or a pharmaceutically acceptable salt, solvate, or polymorph thereof per the weight of the patient. In some embodiments, the patient receives at least about 40 mg/kg of Compound 1, or a pharmaceutically acceptable salt, solvate, or polymorph thereof per the weight of the patient. In some embodiments, the patient receives at least about 50 mg/kg of Compound 1, or a pharmaceutically acceptable salt, solvate, or polymorph thereof per the weight of the patient. In some embodiments, the patient receives at least about 60 mg/kg of Compound 1, or a pharmaceutically acceptable salt, solvate, or polymorph thereof per the weight of the patient. In some embodiments, the patient receives at least about 100 mg/kg of Compound 1, or a pharmaceutically acceptable salt, solvate, or polymorph thereof per the weight of the patient.

In some embodiments, the patient receives Compound 1, or a pharmaceutically acceptable salt, solvate, or polymorph thereof at about 4.0 mg/kg per body weight. In some embodiments, the patient receives Compound 1, or a pharmaceutically acceptable salt, solvate, or polymorph thereof at about 4.5 mg/kg per body weight. In some embodiments, the patient receives Compound 1, or a pharmaceutically acceptable salt, solvate, or polymorph thereof at about 8.0 mg/kg per body weight. In some embodiments, the patient receives Compound 1, or a pharmaceutically acceptable salt, solvate, or polymorph thereof at about 8.5 mg/kg per body weight.

In some embodiments, Compound 1, or a pharmaceutically acceptable salt, solvate, or polymorph thereof is administered to a patient in need thereof at a flat dose of about 100 mg to about 1,000 per day. In some embodiments, Compound 1 is administered at a dose of about 300 to about 800 mg per day.

In some embodiments, Compound 1, or a pharmaceutically acceptable salt, solvate, or polymorph thereof is administered to a patient in need thereof at a flat dose of about 20 mg, about 50 mg, about 100 mg, about 200 mg, about 300 mg, about 400 mg, about 500 mg, about 600 mg, about 700 mg, about 800 mg, about 900 mg, about 1,000 mg, about 1,100 mg, about 1,200 mg, about 1,300, about 1,400, or about 1,500 mg per day. In some embodiments, Compound 1 is administered at about 400 mg per day. In some embodiments, Compound 1 is administered at about 500 mg per day. In some embodiments, Compound 1 is administered at about 600 mg per day. In some embodiments, Compound 1 is administered at about 700 mg per day. In some embodiments, Compound 1 is administered about 800 mg per day. In some embodiments, Compound 1 is administered at about 900 mg per day. In some embodiments, Compound 1 is administered at about 1,000 mg per day. In some embodiments, Compound 1 is administered at about 1,100 mg per day. In some embodiments, Compound 1 is administered at about 1,200 mg per day. In some embodiments, Compound 1 is administered at about 1,300 mg per day. In some embodiments, Compound 1 is administered at about 1,400 mg per day. In some embodiments, Compound 1 is administered at about 1,500 mg per day.

In some embodiments, Compound 1, or a pharmaceutically acceptable salt, solvate, or polymorph thereof is administered to a patient in need thereof at a dose of about 100 mg to about 1,000 per dose. In some embodiments, Compound 1 is administered at a dose of about 300 to about 800 mg per dose.

In some embodiments, Compound 1, or a pharmaceutically acceptable salt, solvate, or polymorph thereof is administered to a patient in need thereof at a about 20 mg, about 50 mg, about 100 mg, about 200 mg, about 300 mg, about 400 mg, about 500 mg, about 600 mg, about 700 mg, about 800 mg, about 900 mg, or about 1,000 mg per dose. In some embodiments, Compound 1 is administered at about 400 mg per dose. In some embodiments, Compound 1 is administered at about 500 mg per dose. In some embodiments, Compound 1 is administered at about 600 mg per dose. In some embodiments, Compound 1 is administered at about 700 mg per dose. In some embodiments, Compound 1 is administered about 800 mg per dose. In some embodiments, Compound 1 is administered at about 900 mg per dose. In some embodiments, Compound 1 is administered at about 1,000 mg per dose.

In some embodiments, Compound 1 or a pharmaceutically acceptable salt, solvate, or polymorph thereof is administered to a patient, wherein Compound 1 is administered once a day (QD).

In some embodiments, Compound 1 or a pharmaceutically acceptable salt, solvate, or polymorph thereof is administered to a patient, wherein Compound 1 is administered twice per day (BID). In some embodiments, Compound 1 or a pharmaceutically acceptable salt, solvate, or polymorph thereof is administered to a patient, wherein Compound 1 is administered three times per day (TID).

In some embodiments, Compound 1, or a pharmaceutically acceptable salt, solvate, or polymorph thereof is administered to a patient, wherein Compound 1, or a pharmaceutically acceptable salt, solvate, or polymorph thereof is administered once a day with food. It has been found that food reduce the adverse event profile dramatically. The incidence and severity of the side effects, such as nausea, emesis and headache, can be reduced when Compound 1, or a pharmaceutically acceptable salt, solvate, or polymorph thereof is taken with food.

In some embodiments, Compound 1, or a pharmaceutically acceptable salt, solvate, or polymorph thereof is administered to a patient, wherein Compound 1, or a pharmaceutically acceptable salt, solvate, or polymorph thereof is administered once a day for at least 7 days, 10 days, 2 weeks, 3 weeks, 4 weeks, 1 month, 2 months, 3 months, 4 months, 6 months, 7 months, 8 months, 9 months, 10 months, 11 months, a year, 1.5 years, or 2 years. In some embodiments, the patients are treated for 3 months. In some embodiments, the patients are treated for 6 months. In some embodiments, the patients are treated for 1 year. In some embodiments, the patients are treated for 1.5 years. In some embodiments, the patients are treated for 2 years, 3 years, 4 years, 5 years, over 5 years, or the duration of life.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

### III. Methods of Treatment

Cardiac or heart failure is a clinical syndrome characterized by a pathophysiological state caused by abnormalities in cardiac structure and/or function that cause a reduction in cardiac output and/or an increase in intracardiac pressure. The heart muscle may become damaged and weakened, and the ventricles stretch or dilate to the point that the heart can no longer pump blood efficiently throughout your body.

In developed countries, the incidence of heart failure is about 1-2% of the adult population, and it rises to more than 10% in people over the age of 70. The lifetime risk of heart failure at the age of 55 is 33% for men and 28% for women. Conditions that can damage or weaken your heart and can cause heart failure include but are not limited to: coronary artery disease, high blood pressure (hypertension), congenital heart defects, faulty heart valves, abnormal heart rhythms arrhythmias), and damage to the heart muscle (cardiomyopathy) from infections, alcohol abuse, obesity, metabolic conditions such as diabetes, and the toxic effect of drugs, such as cocaine or some drugs used for chemotherapy.

Heart failure can involve the left or right ventricle, or both sides of the heart. Generally, heart failure begins with the left side, specifically the left ventricle the heart's main pumping chamber. There are two types of left ventricular heart failure: (1) heart failure with reduced ejection fraction (HFrEF), and (2) heart failure with preserved ejection fraction (HFpEF). An ejection fraction is an important measurement of how well your heart is pumping and is used to help classify heart failure and guide treatment.

Natriuretic peptides are cardiac derived hormones released as a counter-regulatory mechanism to increased cardiovascular stress. The cardioprotective effects of natriuretic peptides occur through the particulate guanylate cyclase receptor to generate the second messenger cGMP, which then acts on target organs to exert anti-proliferative, anti-inflammatory, and anti-adhesion effects, among others to reduce the inciting cardiovascular stress. Not only are natriuretic peptides robust biomarkers of cardiovascular stress and predictors of prognosis, but the physiologic actions of natriuretic peptides are to counteract cardiovascular stress. The cardioprotective effects of natriuretic peptides are mediated via production of cGMP. Phosphodiesterases, in particular PDE9, breakdown natriuretic peptide generated cGMP.

### Cardiac Failure

One aspect of the present disclosure provides for methods of treating cardiac failure to a patient in need thereof, the method comprising administering to the patient a PDE9 inhibitor of Formula (I), or a pharmaceutically acceptable salt, solvate, or polymorph thereof. In some embodiments, the PDE9 inhibitor of Formula (I) is administered at a dose of at least 10 mg/kg per body weight. In some embodiments, the PDE9 inhibitor of Formula (I) is administered at a dose of at least 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg per body weight. In some embodiments, the PDE9 inhibitor of Formula (I) is administered at a dose of about 4.0 mg/kg. In some embodiments, the PDE9 inhibitor of Formula (I) is administered at a dose of about 4.5 mg/kg. In some embodiments, the PDE9 inhibitor of Formula (I) is administered at a dose of about 8.0 mg/kg. In some embodiments, the PDE9 inhibitor of Formula (I) is administered at a dose of about 8.5 mg/kg.

One another aspect of the present disclosure provides for methods of treating cardiac failure to a patient in need thereof, the method comprising administering to the patient a PDE9 inhibitor of Formula (I), or a pharmaceutically acceptable salt, solvate, or polymorph thereof, wherein the PDE9 is administered at a dose of at least 10 mg/kg per body weight. In some embodiments, the PDE9 inhibitor is administered at a dose of at least about 15 mg/kg, at least about 20 mg/kg, at least about 25 mg/kg, at least about 30 mg/kg, at least about 35 mg/kg, at least about 40 mg/kg, at least about 45 mg/kg per body weight, or at least about 50 mg/kg per body weight.

One another aspect of the present disclosure provides for methods of treating cardiac failure to a patient in need thereof, the method comprising administering to the patient a PDE9 inhibitor of Formula (I), or a pharmaceutically acceptable salt, solvate, or polymorph thereof, wherein the PDE9 inhibitor decreases atrial natriuretic peptide (ANP) and/or B-type natriuretic peptide (BNP) prior compared to levels prior to treatment.

In some embodiments, the PDE9 inhibitor of Formula (I) decreases ANP in the subject. In some embodiments, the PDE9 inhibitor of Formula (I) decreases ANP in the subject by at least 5%, 10%, 25%, 50%, 100%, 150%, 200%, or 250% compared to pretreatment levels. In some embodiments, the PDE9 inhibitor of Formula (I) decreases ANP in the subject by at least 5%, 10%, 15%, 20%, 25%, 30%, 35%, 40%, 45%, 50%, 55%, 60%, 65%, 70%, 75%, 80%, 85%, 90%, 95%, or 100% compared to pretreatment levels. In some embodiments, the PDE9 inhibitor of Formula (I) decreases ANP by about 2 times, 3 times, 4 times, 5 times, 10 times, 15 times, 20 times, or 25 times over ANP prior to treatment.

In some embodiments, the PDE9 inhibitor of Formula (I) decreases BNP in the subject. In some embodiments, the PDE9 inhibitor of Formula (I) decreases BNP in the subject by at least 5%, 10%, 25%, 50%, 100%, 150%, 200%, or 250% compared to pretreatment levels. In some embodiments, the PDE9 inhibitor of Formula (I) decreases BNP in the subject by at least 5%, 10%, 15%, 20%, 25%, 30%, 35%, 40%, 45%, 50%, 55%, 60%, 65%, 70%, 75%, 80%, 85%, 90%, 95%, or 100% compared to pretreatment levels. In some embodiments, the PDE9 inhibitor of Formula (I) decreases BNP by about 2 times, 3 times, 4 times, 5 times, 10 times, 15 times, 20 times, or 25 times over BNP prior to treatment.

In some embodiments, the cardiac failure is acute, chronic, or congestive cardiac failure. In some embodiments, the cardiac failure is diabetes induced, autoimmune based, or inflammatory based cardiac failure. In some embodiments, the cardiac failure is with a preserved ejection fraction or with a reduced ejection fraction.

### Cardiac Fibrosis

In another aspect of the present disclosure provides for methods of treating cardiac fibrosis to a patient in need thereof, the method comprising administering to the patient a PDE9 inhibitor of Formula (I), or a pharmaceutically acceptable salt, solvate, or polymorph thereof.

Cardiac fibrosis commonly refers to the excess deposition of extracellular matrix in the cardiac muscle. Fibrocyte cells normally secrete collagen, and function to provide structural support for the heart. When over-activated this process causes thickening and fibrosis of the valve, with white tissue building up primarily on the tricuspid valve, but also occurring on the pulmonary valve. The thickening and loss of flexibility eventually may lead to valvular dysfunction and right-sided heart failure.

In some embodiments, the treating of cardiac fibrosis further comprise decreasing accumulation of fibronectin and/or collagen type I and II. In some embodiments, treatment of cardiac fibrosis further comprises decreasing accumulation of fibronectin. In some embodiments, fibronectin is decreased by at least 5%, 10%, 25%, 50%, 100%, 150%, 200%, or 250%. In some embodiments, fibronectin is decreased by about 2 times, 3 times, 4 times, 5 times, 10 times, 15 times, 20 times, or 25 times over levels prior to treatment. In some embodiments, fibronectin is decreased by about 5%, 10%, 20%, 30%, 40%, or 50%, or more compared to pretreatment levels. In some embodiments, fibronectin is decreased by about 5%, 10%, 15%, 20%, 25%, 30%, 35%, 40%, 45%, or 50%, or more compared to pretreatment levels.

In some embodiments, treatment of cardiac fibrosis further comprises decreasing collagen type I or II. In some embodiments, collagen type I or II is decreased by at least 5%, 10%, 25%, 50%, 100%, 150%, 200%, or 250%. In some embodiments, collagen type I or II is decreased by about 2 times, 3 times, 4 times, 5 times, 10 times, 15 times, 20 times, or 25 times over levels prior to treatment. In some embodiments, collagen type I or II is decreased by about 5%, 10%, 20%, 30%, 40%, or 50%, or more compared to pretreatment levels. In some embodiments, collagen type I or II is decreased by about 5%, 10%, 15%, 20%, 25%, 30%, 35%, 40%, 45%, or 50%, or more compared to pretreatment levels.

### Inflammation

In another aspect of the present disclosure provides for methods of treating myocardial inflammation (myocarditis) to a patient in need thereof, the method comprising administering to the patient a PDE9 inhibitor of Formula (I), or a pharmaceutically acceptable salt, solvate, or polymorph thereof.

Cardiac inflammation or myocarditis is an inflammation of the heart muscle (myocardium). Myocarditis affects both the heart muscle and the heart's electrical system causing rapid or abnormal heart rhythms (arrhythmias). Cardiac inflammation can be caused by infections, particularly from viruses or bacteria; medicines; or damage to the heart's tissue or muscle from autoimmune diseases, medicines, environmental factors, or other triggers. It is most commonly caused by a viruses, and can lead to left-sided heart failure.

In some embodiments, inflammation is reduced by at least 5%, 10%, 25%, 50%, 100%, 150%, 200%, or 250%. In some embodiments, inflammation is reduced the subject by at least 5%, 10%, 15%, 20%, 25%, 30%, 35%, 40%, 45%, 50%, 55%, 60%, 65%, 70%, 75%, 80%, 85%, 90%, 95%, or 100% compared to pretreatment levels.

### Biophysical Effects

Natriuretic peptides play a crucial role in maintaining cardiovascular homeostasis.

Among their properties are vasodilation, natriuresis, diuresis, and inhibition of cardiac remodeling. As heart failure progresses, however, natriuretic peptides fail to compensate. N-terminal pro-B-type natriuretic peptide (NT-proBNP) is used as a diagnostic for diagnosing heart disease and heart failure.

In another embodiment, the PDE9 inhibitor is used increase or decrease biomarkers associated with heart disease, such as atrial natriuretic peptide (ANP) and B-type natriuretic peptide (BNP). In another embodiment, Compound 1 is used increase or decrease biomarkers associated with heart disease, such as atrial natriuretic peptide (ANP) and B-type natriuretic peptide (BNP).

Atrial natriuretic peptide (ANP) hormone of cardiac origin, which is released in response to atrial distension and serves to maintain sodium horeostasis and inhibit activation of the renin-angiotensin-aldosterone system. Congestive heart failure is a clinical syndrome characterized by increased cardiac volume and pressure overload with an inability to excrete a sodium load, which is associated with increased activity of systemic neurohurnoral and local autocrine and paracrine mechanisms. Circulating atrial natriuretic peptide is greatly increased in congestive heart failure as a result of increased synthesis and release of this hormone.

Ventricular natriuretic peptide or B-type natriuretic peptide (BNP), is a hormone secreted by cardiomyocytes in the heart ventricles in response to stretching caused by increased ventricular blood volume. The physiologic actions of BNP are similar to those of ANPs. The net effect of these peptides is a decrease in blood pressure due to the decrease in systemic vascular resistance and, thus, afterload.

In some embodiments, Compound 1 is used to decrease ANP in a subject. ANP may be decreased by at least 5%, 10%, 25%, 50%, 100%, 150%, 200%, or 250%. In some embodiments, Compound 1 is used to decrease ANP by about 2 times, 3 times, 4 times, 5 times, 10 times, 15 times, 20 times, or 25 times over ANP prior to treatment. In some embodiments, the ANP is decrease by about 5%, 10%, 20%, 30%, 40%, or 50%, or more compared to pretreatment levels.

In some embodiments, Compound 1 is used to decrease BNP in a subject. BNP may be decreased by at least 5%, 10%, 25%, 50%, 100%, 150%, 200%, or 250%. In some embodiments, Compound 1 is used to decrease BNP by about 2 times, 3 times, 4 times, 5 times, 10 times, 15 times, 20 times, or 25 times over BNP prior to treatment. In some embodiments, the BNP is decrease by about 5%, 10%, 20%, 30%, 40%, or 50%, or more compared to pretreatment levels.

In another embodiment, Compound 1 is used to increase hemoglobin (Hb) levels in a subject. The Hb level may be increased by at least 5%, 10%, 25%, 50%, 100%, 150%, 200%, or 250%. In some embodiments, Compound 1 is used to increase Hb levels by about 2 times, 3 times, 4 times, 5 times, 10 times, 15 times, 20 times, or 25 times over Hb levels prior to treatment.

In some embodiments, the hemoglobin (Hb) levels of the subject are increased in the range of about 0.5 to about 3.0 g/dL of total Hb. In some embodiments, the hemoglobin (Hb) level of the subject is increased by about 0.5, about 1.0, about 1.5, about 2.0, about 2.5, or about 3.0 g/dL of total Hb.

In another embodiment, Compound 1 is used to increase red blood cell (RBC) levels in a subject. The RBC level may be increased by at least 5%, 10%, 25%, 50%, 100%, 150%, 200%, or 250%. In some embodiments, Compound 1 is used to increase RBC levels by about 2 times, 3 times, 4 times, 5 times, 10 times, 15 times, 20 times, or 25 times over baseline levels prior to treatment.

In yet another embodiment, Compound 1 is used to increase mature RBC levels, reduce immature RBC levels, and/or increase maturation ratio. RBC maturation is measured by calculating the ratio of immature red blood cells (RBC) (Ery.B: late basophilic and polychromatic) in relation to mature RBC (Ery.C: ortochromatic and reticulocytes) i.e. as Ery.B/Ery.C. The mature RBC level may be increased by at least 5%, 10%, 25%, 50%, 100%, 150%, 200%, or 250%. In some embodiments, mature RBC level is increased by about 2 times, 3 times, 4 times, 5 times, 10 times, 15 times, 20 times, or 25 times over the baseline level prior to treatment. The immature RBC level may be reduced by at least 5%, 10%, 20%, 30%, 40%, 50%, 60%, 70%, or 80%. The maturation ratio may be increased by at least 5%, 15%, 25%, 50%, 100%, 150%, 200%, or 250%. In some embodiments, the maturation ratio is increase by about 2 times, 3 times, 4 times, 5 times, 10 times, 15 times, 20 times, or 25 times over the baseline ratio prior to treatment.

### Combination Therapies

Another aspect of the present disclosure provides methods of using the PDE9 inhibitor of the present disclosure, such as Compound 1, or a pharmaceutically acceptable salt, solvate, or polymorph thereof, in combination with at least one other active agent. They may be administered simultaneously or sequentially. They may be present as a mixture for simultaneous administration, or may each be present in separate containers for sequential administration.

In another embodiment of the present invention, the one or more additional therapeutic agents are one or more of angiotensin transferase inhibitors (ACEIs), β-receptor blockers, mineralocorticoid/aldosterone receptor antagonists (MRAs), diuretics, angiotensin receptor neprilysin inhibitors (ARNIs), neprilysin inhibitors (NEPIs), If channel inhibitors, angiotensin II receptor blockers (ARBs), positive inotropic agents, vasodilator agents, and hydralazines (HYDs) or isosorbide dinitrates (SNDs).

In another embodiment of the present invention, for the second or more therapeutic agents, the ACEIs include but are not limited to: captopril, enalapril, lisinopril, and trandolapril; the f3-receptor blockers include but are not limited to: bisoprolol, carvedilol, metoprolol succinate, and nebivolol; the MRAs include but are not limited to: eplerenone and spirolactone; the ARNIs include: sacubitril/valsartan; the NEPIs include but are not limited to sacubitril; the II channel inhibitors include but are not limited to: ivabradine; ARBs include but are not limited to: candesartan and valsartan; the positive inotropic agents include but are not limited to: digitalis cardiac glycosides such as digoxin or deslanoside, f3-adrenergic receptor agonists such as dopamine or dobutamine or dopexamine, phosphodiesterase inhibitors such as milrinone or amrinone or enoximone, phosphocreatine or cyclohexylethylamine and other positive inotropic agents; the vasodilator agents include but are not limited to: nitroglycerin, isosorbide dinitrate, sodium nitroprusside, and prazosin; and the diuretics include but are not limited to: furosemide, bumetanide, torasemide, bendrofluazide, hydrochlorothiazide, metolazone, indapamide, amiloride, and triamterene.

In some embodiments, the additional active agent is a beta blocker (carvedilol, metoprolol, bisoprolol), an ACE inhibitor (enalapril (Vasotec), lisinopril (Zestril) and captopril (Capoten)), an angiotensin receptor blocker (losartan), an aldosterone antagonist (spironolactone (Aldactone) and eplerenone (Inspra)), digoxin (lanoxin), diuretics (furosemide (Lasix)), or ARC inhibitor (losartan (Cozaar) and valsartan (Diovan)).

In some embodiments, the additional therapeutic is hydroxy urea (HU).

The other active agent may be a different PDE9 inhibitor of the present disclosure. The other active agent may also be an antibiotic agent such as penicillin, a nonsteroidal anti-inflammatory drug (NSAIDS) such as diclofenac or naproxen, a pain relief medication such as opioid, or folic acid. In some embodiments, the other active agent is folic acid.

The term “simultaneous administration”, as used herein, is not specifically restricted and means that the PDE9 inhibitor of the present disclosure and the at least one other active agent are substantially administered at the same time, e.g. as a mixture or in immediate subsequent sequence.

The term “sequential administration”, as used herein, is not specifically restricted and means that the PDE9 inhibitor of the present disclosure and the at least one other active agent are not administered at the same time but one after the other, or in groups, with a specific time interval between administrations. The time interval may be the same or different between the respective administrations of PDE9 inhibitor of the present disclosure and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the PDE9 inhibitor of the present disclosure and the at least one additional active agent is not particularly restricted. For example, when a PDE9 inhibitor of the present disclosure and the one other additional active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when a PDE9 inhibitor of the present disclosure and two or more other active agents are combined in a composition. The PDE9 inhibitor of the present disclosure may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

### IV. Kits and Devices

The disclosure provides a variety of kits and devices for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for treating heart disease, comprising a PDE9 inhibitor compound of the present disclosure or a combination of PDE9 inhibitor compounds of the present disclosure, optionally in combination with any other active agents, such as folic acid, an antibiotic agent such as penicillin, a nonsteroidal anti-inflammatory drug (NSAIDS) such as diclofenac or naproxen, a pain relief medication such as opioid, or folic acid.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of PDE9 inhibitor compounds in the buffer solution over a period of time and/or under a variety of conditions.

The present disclosure provides for devices that may incorporate PDE9 inhibitor compounds of the present disclosure. These devices contain in a stable formulation available to be immediately delivered to a subject in need thereof, such as a human patient with cardiac failure or cardiac fibrosis.

Non-limiting examples of the devices include a pump, a catheter, a needle, a transdermal patch, a pressurized olfactory delivery device, iontophoresis devices, multi-layered microfluidic devices. The devices may be employed to deliver PDE9 inhibitor compounds of the present disclosure according to single, multi- or split-dosing regiments. The devices may be employed to deliver PDE9 inhibitor compounds of the present disclosure across biological tissue, intradermal, subcutaneously, or intramuscularly. More examples of devices suitable for delivering PDE9 inhibitor compounds include but not limited to a medical device for intravesical drug delivery disclosed in International Publication WO 2014036555, a glass bottle made of type I glass disclosed in U.S. Publication No. 20080108697, a drug-eluting device comprising a film made of a degradable polymer and an active agent as disclosed in U.S. Publication No. 20140308336, an infusion device having an injection micropump, or a container containing a pharmaceutically stable preparation of an active agent as disclosed in U.S. Pat. No. 5,716,988, an implantable device comprising a reservoir and a channeled member in fluid communication with the reservoir as disclosed in International Publication WO 2015023557, a hollow-fiber-based biocompatible drug delivery device with one or more layers as disclosed in U.S. Publication No. 20090220612, an implantable device for drug delivery including an elongated, flexible device having a housing defining a reservoir that contains a drug in solid or semi-solid form as disclosed in International Publication WO 2013170069, a bioresorbable implant device disclosed in U.S. Pat. No. 7,326,421, contents of each of which are incorporated herein by reference in their entirety.

### V. Definitions

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures.

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to amelioration of a disease or disorder, or at least one sign or symptom thereof. “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

As used herein, “prevention” or “preventing” refers to a reduction of the risk of acquiring or having a sign or symptom a given disease or disorder, i.e., prophylactic treatment.

The phrase “therapeutically effective amount” as used herein means that amount of a compound, material, or composition comprising a compound of the present teachings that is effective for producing a desired therapeutic effect. Accordingly, a therapeutically effective amount treats or prevents a disease or a disorder, e.g., ameliorates at least one sign or symptom of the disorder. In various embodiments, the disease or disorder is a cancer.

A dash (“-”) that is not between two letters or symbols is used to indicate a point of attachment for a substituent. For example, —CONH₂ is attached through the carbon atom (C).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂- C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

### List of Abbreviations and Terms

¹H-NMR: Proton Nuclear Magnetic Resonance spectroscopy
ADME: Absorption, Distribution, Metabolism, and Excretion
AE: adverse event
AUC₀₋₂₄: area under the concentration-time curve from time 0 to 24 hours post-dose
BBB: blood-brain barrier
Cₘₐₓ: maximum plasma concentration
cGMP: cyclic guanosine monophosphate
CNS: central nervous system
CV: coefficient of variation
CYP: cytochrome p450
DMC: Data Monitoring Committee
DMSO: dimethyl sulfoxide
DOAC: direct-acting oral anti-coagulant
ECG: electrocardiogram
EOT: end of treatment
FIH: first in human
FTIR: Fourier transform infrared spectroscopy
GC: gas chromatography
hERG: human ether-à-go-go related gene
HPLC: high-performance liquid chromatography
HU: hydroxyurea
IC: inhibitory concentration
IC₅₀: a half minimal inhibitory concentration
IV: intravenous
MAD: multiple-ascending dose
MTD: maximum tolerated dose
NO: nitric oxide
NOAEL: no-observed-adverse-effect level
PD: pharmacodynamic
PDE9: phosphodiester-9
PEG polyethylene glycol
P-gp: P-glycoprotein
PIC: Powder in capsule
PK: pharmacokinetic(s)
RBC: red blood cell
RH: relative humidity
qd: once daily
QoL: quality of life
SAD: single ascending dose
SAE: serious adverse event
SD: standard deviation
SEM: standard error of the mean
sGC: soluble guanylyl cyclase
t_{1/2}: half-life
TK: Toxicokinetic
Tₘₐₓ: time of maximum concentration
ULN: upper limit of normal
WBC: white blood cell
w/w %: weight/weight percent

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### Example 1. Synthesis and Formulation of Compound 1

Compound 1 is an enantiomer of 6-[4-methyl-1-(pyrimidin-2-ylmethyl)pyrrolidin-3-yl]-3-tetrahydropyran-4-yl-7H-imidazo[1,5-a]pyrazin-8-one disclosed in WO 2013/053690. Compound 1 may be prepared from chiral-selective purification from 6-[4-methyl-1-(pyrimidin-2-ylmethyl)pyrrolidin-3-yl]-3-tetrahydropyran-4-yl-7H-imidazo[1,5-a]pyrazin-8-one prepared according to the method disclosed in WO 2013/053690, the contents of which are incorporated herein by reference in their entirety. Compound 1 may also be prepared with the method disclosed in WO 2017/005786, the contents of which are incorporated herein by reference in their entirety. Compound 1 is also named IMR-687.

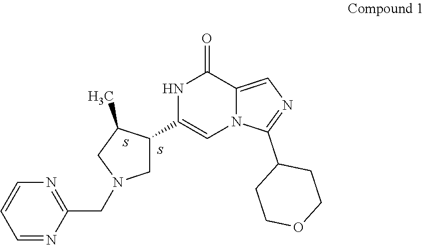

### Example 2. Effects of Compound 1 on Hypertension-Induced Heart Failure with Preserved Ejection Fraction (HFpEF) Models

Systemic hypertension is the single most important comorbidity seen in HFpEF, with a prevalence of 60% to 89% reported from large controlled trials, epidemiological studies, and heart failure (HF) registries. Increased blood pressure induces cardiomyocyte and fibroblast changes and accelerates cardiac remodeling. Moreover, hypertension results in vascular changes such as endothelial dysfunction, reduced coronary reserve blood flow, and diminished capillary density, all of which lead to reduced oxygen delivery. Systemic hypertension also results in arterial stiffness, which imposes a disproportionate load on the heart, leading to ventricular-vascular uncoupling and afterload mismatch. These changes lead to impaired systolic and diastolic function.

### Aldosterone-Infused and Unilateral Nephrectomized Mouse

Mice subjected to uninephrectomy and aldosterone infusion for 4 to 6 weeks, accompanied by 1% NaCl intake, develop HFpEF with moderate hypertension, concentric left ventricle hypertrophy, pulmonary congestion, and echocardiographic evidence of diastolic dysfunction while maintaining a normal/preserved LVEF. These mice also show exercise impairment. At the molecular level, left ventricle tissue from these mice show an increase in natriuretic peptides, cardiac size and fibrosis, as well as an increase in the oxidative stress.

### Angiotensin II-Infused Mouse

Administration of angiotensin II for a variable timeframe (1 to 8 weeks) in mice leads to cardiac hypertrophy and remodeling, both in the presence and absence of hypertension, suggesting that cardiac remodeling under angiotensin II infusion is due to blood pressure-dependent and independent factors. C57BL/6J mice develop compensatory concentric hypertrophy and fibrosis in response to angiotensin II. Pulmonary congestion, as well as exercise intolerance, are evident and seem to be related to angiotensin II-induced skeletal muscle abnormalities, including impaired mitochondrial function and skeletal muscle atrophy. In summary, if strain and dosage are optimized to mirror the human HFpEF phenotype, angiotensin II infusion appears to be a relevant HFpEF model.

### Example 3. Effects of Compound 1 on Obesity and Diabetes-Induced Heart Failure with Preserved Ejection Fraction (HFpEF) Models

Obesity induces significant structural changes in the left ventricle, and patients with HFpEF are significantly more likely to be obese. There are multiple mechanisms whereby obesity could contribute to HFpEF. Increased adiposity promotes inflammation, insulin resistance, and dyslipidemia and also impairs arterial, skeletal muscle, and physical function, all of which are abnormal in patients with HFpEF. Diabetes is also commonly seen in HFpEF. Systemic insulin resistance and hyperglycemia trigger cardiac insulin resistance and neurohormonal, sympathetic, and cytokine imbalance in the heart. This, in turn, might induce cardiac remodeling processes such as cardiomyocyte hypertrophy, interstitial fibrosis and collagen modifications, leading to further cell damage and deterioration of diastolic and systolic function.

db/db Mouse

The db/db leptin receptor-deficient mouse has a point mutation in the diabetes (db) gene encoding the leptin receptor, which spontaneously causes morbid obesity accompanied by severe hyperglycemia secondary to type 2 diabetes. Thus, this model is valuable in exploring the combined contribution of obesity and type 2 diabetes to HFpEF, which is representative of this particular HFpEF phenotype. db/db mice show an inflammatory, systemic cytokine fingerprint and despite the presence of both hyperinsulinemia and hyperleptinemia, mice do not initially show cardiac hypertrophy, but it eventually develops at older ages (6 months). At the histological level, these mice hearts have enlarged cardiomyocytes, evidence of fibrosis, and capillary rarefaction. The db/db mice appear to represent the obese/metabolic HFpEF phenotype, with evidence of HF, whereas LVEF is preserved.

### Example 4. Role of Compound 1 in HFPEF Pre-Clinical Models to Improve Heart Function and Decrease Myocardial Hypertrophy, Fibrosis, and Inflammation

Model 1: eight-week-old C57BL/6J male mice were anesthetized with 100 mg/kg ketamine and 10 mg/kg xylazine intraperitoneally. All mice underwent unilateral nephrectomy and then will receive a continuous infusion of d-aldosterone (0.30 μg/h) via osmotic minipumps. All mice were maintained on 1.0% sodium chloride drinking water. Mice were randomized to receive either vehicle or Compound 1 at 60 mg/kg/day or 100 mg/kg/day by oral gavage for 4 or 6 weeks (36 mice, N=6/group). The mice were housed in a standard-temperature, 12 h/12 h light/dark controlled room, with water and standard rodent diet available ad libitum. Mice were euthanized 4 or 6 weeks after the beginning of the treatment. It has been shown that administering of 60 mg/kg of Compound 1 in mice is equivalent to administering about 4.8 mg/kg of Compound 1 in adult human subjects. Similarly, it has been shown that administering 100 mg/kg of Compound 1 in mice is equivalent to administering about 8.1 mg/kg of Compound 1 in human subjects.

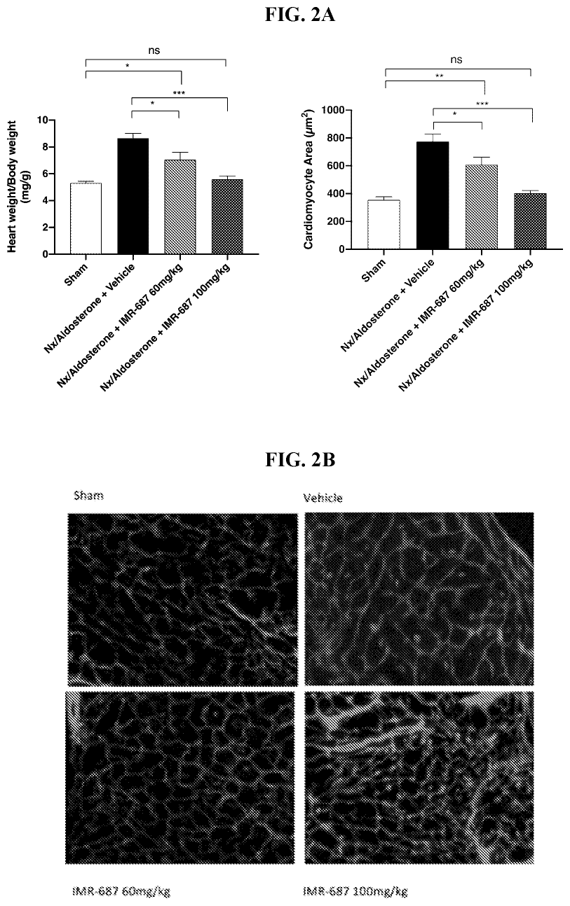

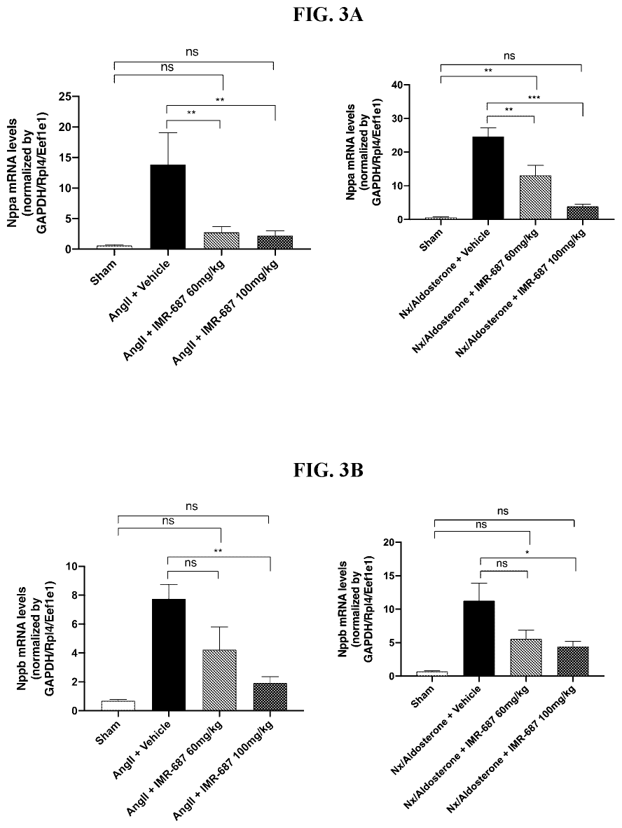

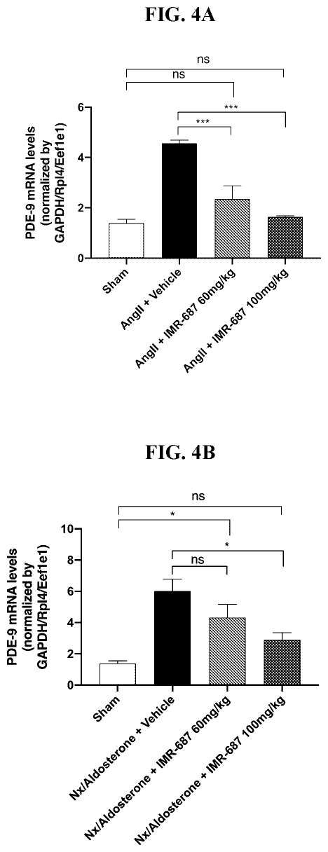

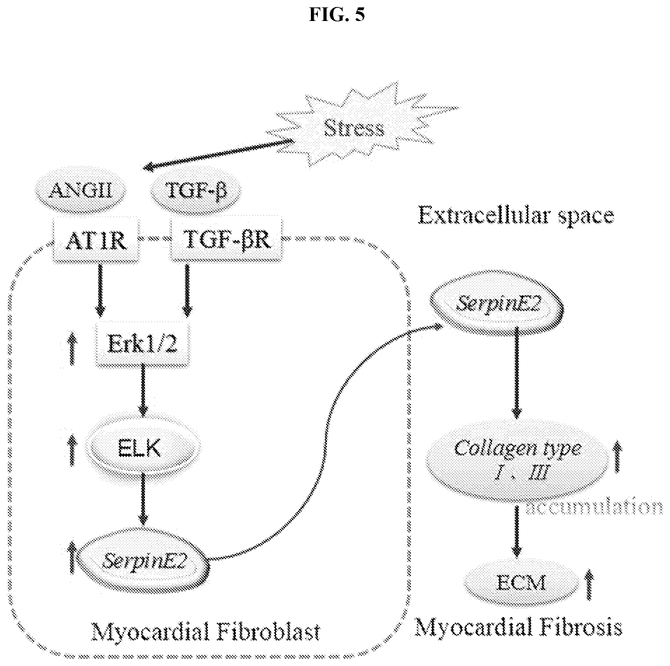

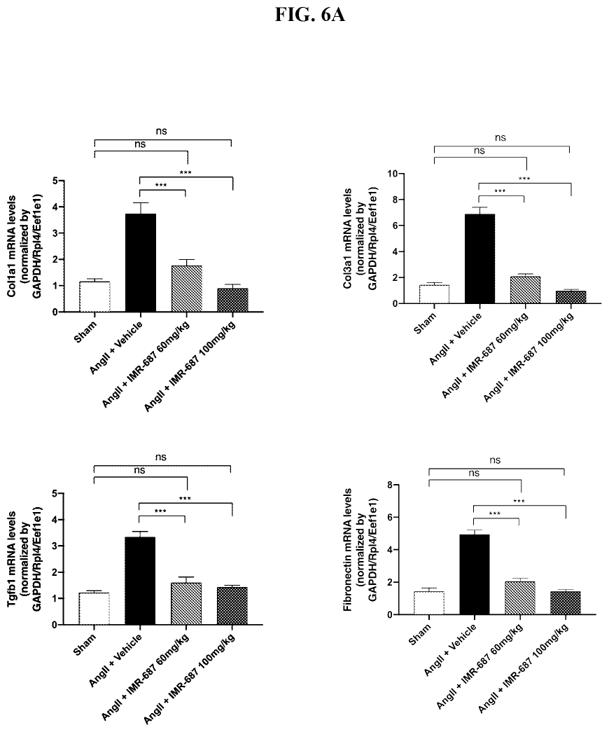

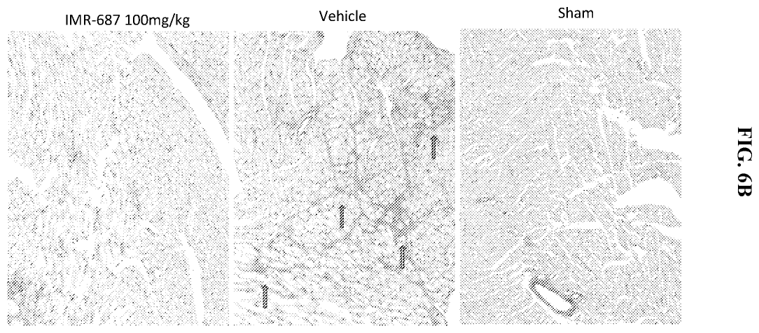

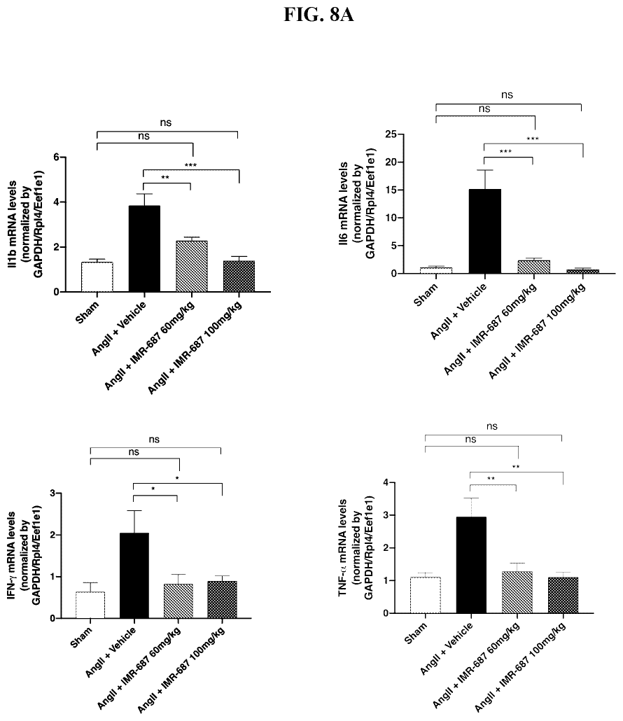

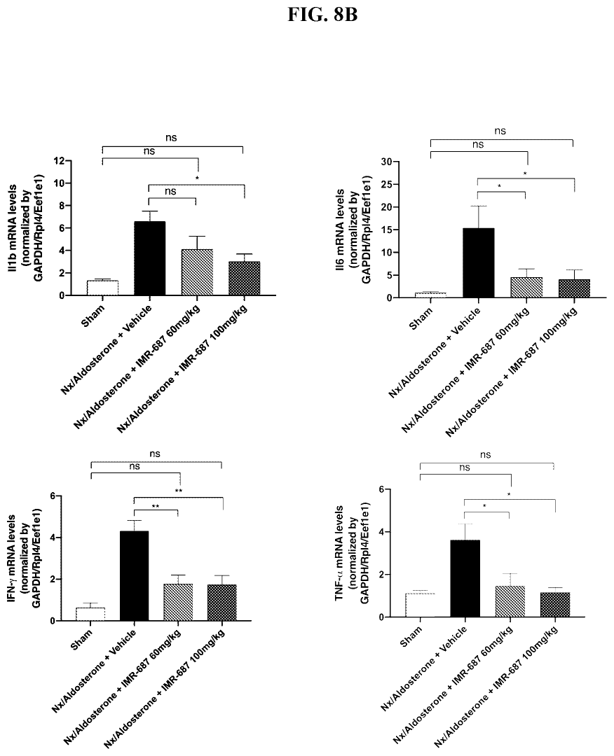

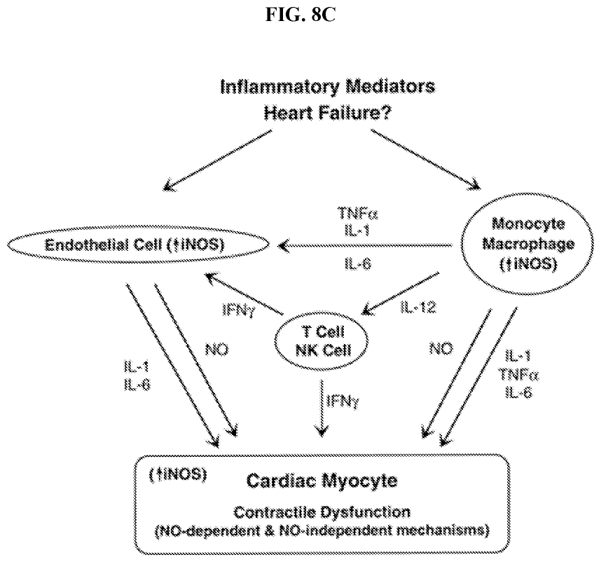

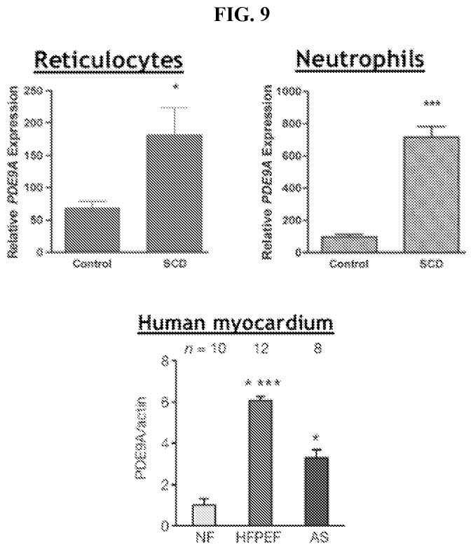

Several biological effects were tested, including: heart size and cardiomyocyte hypertrophy (FIGS. 2A and 2B); ANP and BNP levels (FIGS. 3A right side, and 3B right side); PDE-9 mRNA expression levels (FIG. 4B); TGF-β1 levels and downstream targets (Fibronectin and Collagen type I and III) (FIG. 7); and myocardial inflammation biomarkers (FIG. 8B). Collectively, this data shows Compound 1 (with or without nephrectomy and a continuous infusion of d-aldosterone) is effective in treating various forms of heart disease (e.g. myocardial inflammation, fibrosis, etc.)

Model 2: eight-week-old C57BL/6J male mice were anesthetized with isoflurane and subcutaneously implanted with an osmotic minipump to continuously infuse angiotensin II in 10 mM acetic acid at a dose of 1.5 mg/kg per day over a period of 4 to 6 weeks. Experimental animal groups were randomly assigned to receive either vehicle or Compound 1 at 60 mg/kg/day or 100 mg/kg/day by oral gavage for 4 or 6 weeks (36 mice, N=6/group) over the same time period. The mice were housed in a standard-temperature, 12 h/12 h light/dark controlled room, with water and standard rodent diet available ad libitum. Mice were euthanized 4 or 6 weeks after angiotensin-II infusion. It has been shown that administering of 60 mg/kg of Compound 1 in mice is equivalent to administering about 4.8 mg/kg of Compound 1 in adult human subjects. Similarly, it has been shown that administering 100 mg/kg of Compound 1 in mice is equivalent to administering about 8.1 mg/kg of Compound 1 in human subjects.

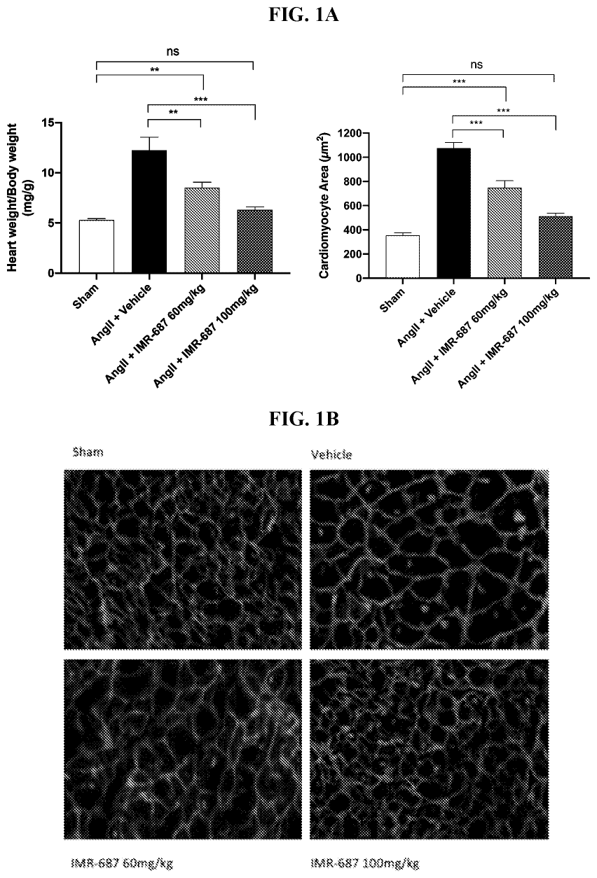

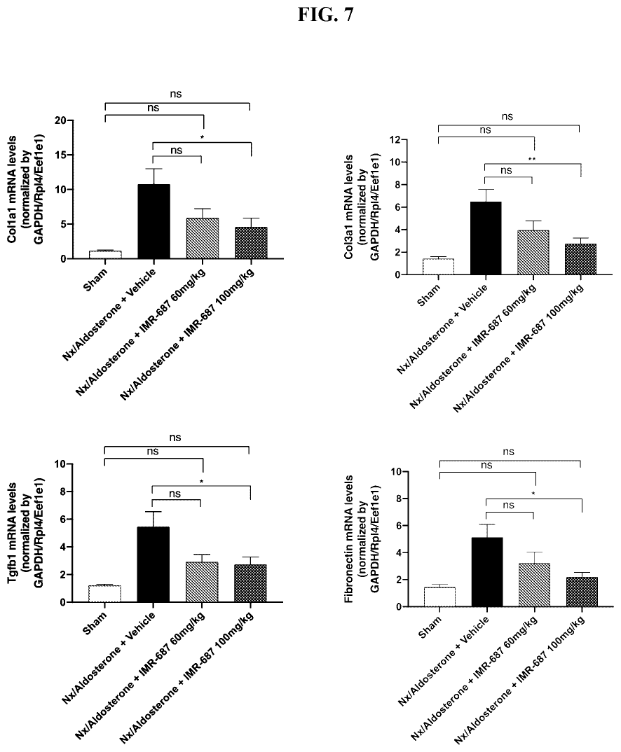

Several biological effects were tested, including: heart size and cardiomyocyte hypertrophy (FIGS. 1A and 1B); ANP and BNP levels (FIGS. 3A left side, and 3B left side); PDE-9 mRNA expression levels (FIG. 4A); TGF-β1 levels and downstream targets (Fibronectin and Collagen type I and III) (FIG. 6A); and myocardial inflammation biomarkers (FIG. 8A). Collectively, this data shows Compound 1 (with or without nephrectomy and a continuous infusion of angiotensin II) is effective in treating various forms of heart disease (e.g. cardiac failure, cardiac fibrosis, myocardial inflammation, etc.).

### Example 5. Role of Compound 1 in HFpEF Pre-Clinical Models to Improve Weight Loss in Diabetes-Related and Non-Diabetes Related Obesity

Model 3: twenty-week old male diabetic-prone, obese db/db mice of the BKS.Cg-Dock7m+/+Leprdb/J strain will be randomly assigned (30 mice, n=10/group) to receive vehicle or chronic Compound 1 treatment at 60 mg/kg/day or 100 mg/kg/day. Treatment will be carried for 8 weeks using subcutaneous osmotic pumps. The mice will be housed in a standard-temperature, 12 h/12 h light/dark controlled room, with water and standard rodent diet available ad libitum.

Tasks deliverables: Using mouse models of HFpEF treated with Compound 1 at 60 mg/kg/day or 100 mg/kg/day we propose to evaluate:
(a) Physiological measurements: weekly monitoring of heart rate (bpm) and blood pressure (mmHg) using a non-invasive tail-cuff blood pressure analyzer. Insulin and glucose levels at beginning of treatment (day 0) and at the day of the sacrifice (4 or 6 weeks for model #1 and model #2, 12 weeks for model #3).
(b) Body weight (g), heart weight/body weight (mg/g), serum aldosterone levels and serum angiotensin-II levels by ELISA.
(c) Myocardial cGMP concentration using a parameter cGMP immunoassay.
(d) Left ventricular cardiomyocyte hypertrophy: atrial natriuretic peptide and brain natriuretic peptide mRNA expression (encoded by the genes Nppa and Nppb), cardiomyocyte cross-sectional area in sections stained with hematoxylin-eosin, cardiomyocyte cell diameter in sections stained with wheat germ agglutinin.
(e) Myocardial PDE-9 expression: mRNA by real-time PCR and protein by Western Blot.
(f) Myocardial Fibrosis: fibrosis area (%) in sections stained with Masson Trichrome and PAS, mRNA expression of collagen type I and type III, fibronectin and TGF-beta1 by real-time PCR.
(g) Myocardial Oxidative Stress: immunohistochemical analysis of sections stained with 3-nitrotyrosine antibodies.
(h) Calcium-handling proteins and signaling pathways: immunoblotting of sarcoplasmic reticulum Ca²⁺-ATPase (SERCA2a), Ca²⁺/calmodulin-dependent protein kinase II (CaMKII), protein kinase A (PKA) and phospholamban (total, phosphor-Ser16 and phosphor-Thr17).
(i) Myocardial inflammation: immunohistochemical analysis of macrophage infiltration, cytokine mRNA expression (IL-1b, IL-6, IL-8, IL-13, IL-17, IFN-gamma, TNF-alpha) by real-time PCR, inflammation markers on cardiomyocytes and plasma by proteome profiler and bioplex assay.
(j) Markers of endothelial activation: plasma levels of soluble E-selectin (CD62E), P-selctin (CD62P), vascular adhesion molecule 1 (VCAM-1) and intercellular adhesion molecule 1 (ICAM-1) by ELISA.
(k) Lung congestion: wet-lung weight/dry lung weight, histological analysis of lung sections stained with hematoxylin-eosin.
(l) Renal injury and fibrosis: histological analysis of kidney sections stained with hematoxylin-eosin and fibrosis area (%) in sections stained with Masson Trichrome and PAS; mRNA expression of collagen type I and type III, fibronectin and TGF-beta1 by real-time PCR. Renal function will be assessed by levels of plasma creatinine, blood urea nitrogen (BUN), urinary NGAL and albuminuria.
(k) Model #3 (db/db): determination of weight loss by comparing the body weight in the beginning and the end of treatment; NPR-C adipose tissue mRNA expression by real-time PCR; accumulation of adipose tissue macrophages by immunohistochemistry.

### Example 6. Phosphodiesterase-9 Inhibition with IMR-687 and Natriuretic Peptide Levels in Adult Patients with Sickle Cell Disease

IMR-SCD-102 is an ongoing Phase 2a randomized double-blind placebo-controlled trial testing Compound 1 (IMR-687) at 50 mg-200 mg daily as monotherapy or 50-100 mg in combination with background hydroxyurea therapy (Compound 1+HU). A protocol amendment later in the study allowed for sample collection and characterization of NT-proBNP levels in the combination cohort (Compound 1+HU versus HU alone). Plasma NT-proBNP was measured in 15 subjects (100% HbSS genotype) at randomization and again at 4 months. A 2:1 randomization schema favoring combination treatment translated to 10 subjects on Compound 1+HU and 5 on HU alone. Baseline, 4-month follow-up, and change in NT-proBNP levels were quantified. Further, whether the change in NT-proBNP level varied according to treatment and baseline NT-proBNP level was tested.

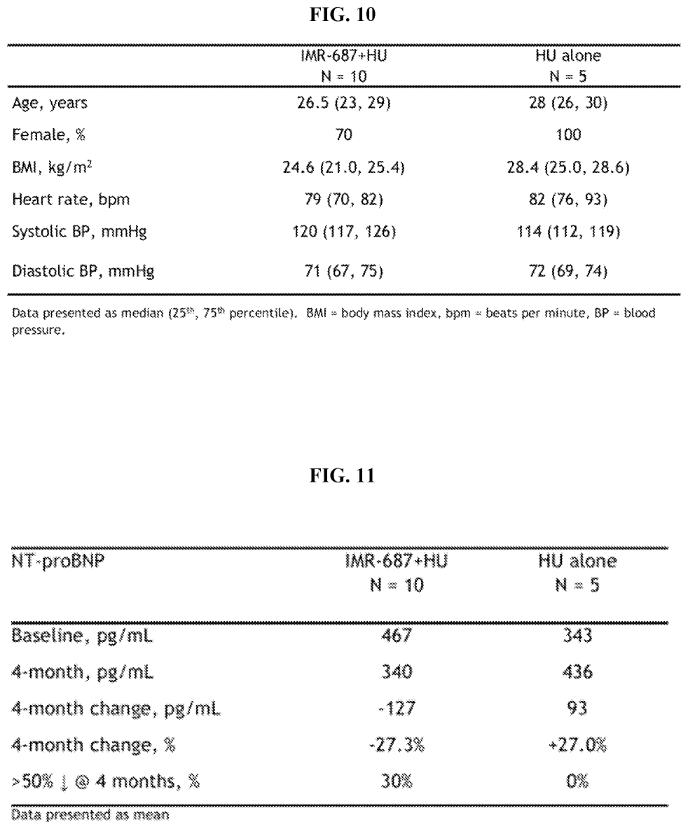

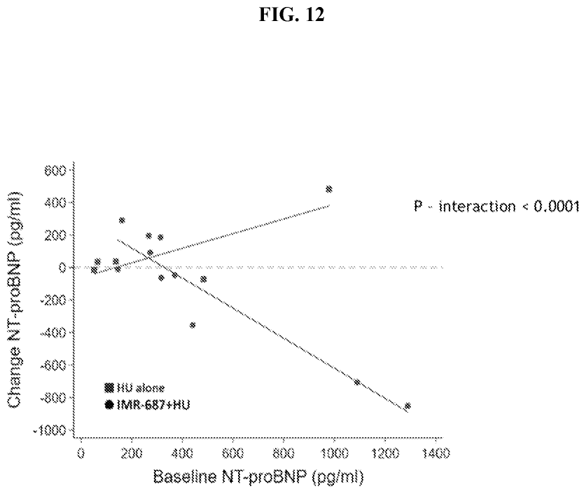

Baseline characteristics of subjects randomized to Compound 1+HU or HU alone were similar (FIG. 10). Mean NT-proBNP levels are also analyzed (FIG. 11). In the Compound 1+HU group, the mean baseline and 4-month follow-up NT-proBNP levels were 467 and 340 pg/ml, respectively (mean decrease of 127 pg/ml or 27.3% reduction). In the HU group, the mean baseline and 4-month follow-up NT-proBNP levels were 343 and 436 pg/ml, respectively (mean increase of 93 pg/ml or 27.0% higher). A greater than 50% reduction in NT-proBNP levels were seen at 4-months in 30% of Compound 1+HU treated subjects, but none of the HU alone treated subjects. For the 4-month change in NT-proBNP, the main effect of Compound 1+HU was significant (p=0.01), but the interaction effect of Compound 1+HU by baseline NT-proBNP level was highly significant (p<0.0001) (FIG. 12). In subjects with baseline NT-proBNP values ≥400 pg/ml, Compound 1+HU was associated with an average 67.9% reduction in NT-proBNP between baseline and 4 months compared with an average 28.0% increase with HU alone. Among subjects with baseline NT-proBNP levels <400 pg/ml, 4-month treatment with Compound 1+HU did not significantly change NT-proBNP levels compared with HU alone. Compound 1+HU combination was not associated with changes in heart rate or blood pressure over 4 months compared with HU alone.

The addition of Compound 1 to HU treated subjects appears to have a favorable cardiovascular safety profile with potential efficacy in reducing cardiovascular risk among adults with SCD, particularly those with baseline NT-proBNP levels ≥400 pg/ml.

### Example 7. Selective PDE9 Inhibition with Compound 1 Mitigates Cardiac Hypertrophy and Renal Injury in Preclinical Mouse Models of Heart Failure with Preserved Ejection Fraction

Introduction: Through degradation of the cardio- and renal-protective second messenger cyclic GMP, PDE9 excess may contribute to cardiomyocyte hypertrophy, fibrosis, and renal dysfunction, common features in heart failure with preserved ejection fraction (HFpEF) development and progression.

Selective PDE9 inhibition with Compound 1 mitigates an adverse cardiac and renal phenotype in mouse models of HFpEF.

Methods: Cardiac and renal responses to Compound 1 (60 mg/kg, 100 mg/kg) compared with vehicle were examined over 6-8 weeks in 3 adult male mouse models of HFpEF (1.5 mg/kg/d angiotensin-II infusion [ang-II]; uninephrectomy+0.30 μg/h d-aldosterone infusion+1% NaCl drinking water [neph-aldo]; and db/db [db]). Phenotyping included wheat germ agglutinin staining for cardiomyocyte cross-sectional area (CSA); RT-PCR for myocardial PDE9, natriuretic peptide, inflammatory and fibrosis marker transcript abundances; ELISA for plasma natriuretic peptides; and urinary albumin to creatinine ratio (UACR).

Results: Compound 1 reduced median cardiomyocyte size (CSA) by 54, 58, and 35% compared with vehicle in the ang-II, neph-aldo, and db models, respectively; p<0.003 for all. Myocardial PDE9, NPPA, NPPB, COL3A1, and IL-1 expression were decreased by Compound 1 in all models (Table 1). Median plasma BNP levels (pg/mL) were lower in Compound 1 vs. vehicle treated mice in all models (ang-II: 2376 vs. 5757; neph-aldo: 1216 vs. 1860; db: 830 vs. 1216); p<0.007 for all, with similar findings for ANP (Table 1). UACR was lower in Compound 1 compared with vehicle treated mice in all models (Table 1). Heart rate and blood pressure did not differ between Compound 1 and vehicle treated mice. Results are shown in Table 1.

TABLE 1
Cardiac and renal responses to Compound 1 compared
with vehicle in adult male mouse models of HFpEF.

| | Ang-II | | | Neph + Aldo | | | db/db | | |
|---|---|---|---|---|---|---|---|---|---|
| | Vehicle | Cmpd 1 | | Vehicle | Cmpd 1 | | Vehicle | Cmpd 1 | |
| | N = 5 | N = 12 | p | N = 6 | N = 12 | p | N = 7 | N = 16 | p |
| Myocardial | | | | | | | | | |
| mRNA | | | | | | | | | |
| PDE9 | 4.6 | 1.7 | 0.003 | 5.9 | 3.7 | 0.024 | 2.5 | 1.5 | 0.001 |
| NPPA | 9.4 | 1.4 | 0.002 | 21.9 | 4.9 | 0.001 | 3.9 | 1.7 | 0.004 |
| NPPB | 7.4 | 2.0 | 0.020 | 11.2 | 4.2 | 0.005 | 5.9 | 2.2 | 0.004 |
| COL3A1 | 6.9 | 1.5 | 0.002 | 7.6 | 2.6 | 0.005 | 2.7 | 1.1 | 0.008 |
| IL-1b | 3.9 | 1.9 | 0.003 | 5.4 | 2.5 | 0.025 | 10.0 | 3.6 | 0.003 |
| Plasma | | | | | | | | | |
| BNP, pg/mL | 5757 | 2376 | 0.006 | 1860 | 1216 | 0.002 | 1216 | 830 | <0.001 |
| ANP, pg/mL | 2759 | 968 | 0.003 | 1267 | 740 | 0.001 | 1106 | 714 | 0.001 |
| Urine | | | | | | | | | |
| UACR, μg/mg | 169.7 | 95.5 | 0.011 | 97.9 | 48.4 | 0.049 | 219.9 | 132.5 | 0.001 |

Median values displayed. P-value from Wilcoxon rank-sum test. Ang-II model (8-week old C57BL/6 mice were infused with ang-II at 1.5 mg/h for 6 weeks with concomitant Compound 1 or vehicle). Neph+d-aldo+1% NaCl model (8-week old C57BL/6 mice underwent uninephrectomy then were infused with d-aldosterone for 6 weeks with ad-libitum 1% NaCl drinking water while receiving concomitant Compound 1 or vehicle). db model (20-week old BKS.Cg-Dock7m+/+Leprdb/J received vehicle or Compound 1 for 8 weeks). mRNA expression levels were normalized to GAPDH, Rpl4 and Eef1e1.

Conclusion: Selective PDE9 inhibition with Compound 1 was effective for prevention and treatment of cardiac hypertrophy and renal dysfunction in three different preclinical models of HFpEF. Compound 1 is a promising candidate therapy for testing in clinical trials of human HFpEF.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating cardiac failure in a patient in need thereof, the method comprising administering a pharmaceutically acceptable dose to the patient of a PDE9 inhibitor of Formula (I), or a pharmaceutically acceptable salt or solvate thereof: 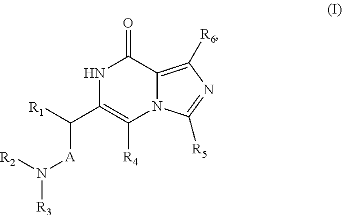
wherein R² is cyclized with either R¹ or R³;
wherein R¹, R², and R³ are
R¹, when cyclized with R² is 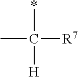
wherein R⁷ is selected from the group consisting of H, —CH₃, —C₂H₅, and C₃H₇;
wherein * denotes the cyclization point; and
R¹, when not cyclized, is selected from the group consisting of H and 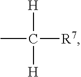
wherein R⁷ is selected from the group consisting of H, —CH₃, —C₂H₅, and C₃H₇;
R² is a compound selected from the group consisting of 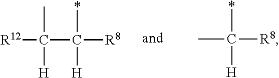
wherein R⁸ and R¹² independently are selected from the group consisting of H, —CH₃, —C₂H₆, and —C₃H₇,
wherein * denotes the cyclization point; and
R³, when cyclized with R² is 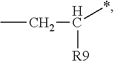
wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R³, when not cyclized, is 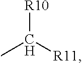
wherein
R¹⁰ is selected from the group consisting of H, —CH₃, and —C₂H₅; and
R¹¹ is selected from the group consisting of C₆-C₁₉ aryl, substituted C₆-C₁₀ aryl, C₃-C₉ heteroaryl, substituted C3-C9 heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C3-C6 heterocyclyl, C₃-C₆ cycloalkyl, and substituted C₃-C₆ cycloalkyl;
R⁶ is selected from the group consisting of hydrogen, F, Cl, CN, —CH₃, —C₂H₅, —C₃H₇, and —CF₃; and
A is absent or —CH₂; and
wherein the PDE9 inhibitor of Formula (I) is administered at a dose of less than or greater than 10 mg/kg per body weight.

2. The method of claim 1, wherein the PDE9 inhibitor of Formula (I) is 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt or solvate thereof.

3. The method of claim 1 or 2, wherein the cardiac failure is acute, chronic, or congestive cardiac failure.

4. The method of any one of claims 1-3, wherein the cardiac failure is diabetes induced, autoimmune based, or inflammatory based cardiac failure.

5. The method of any one of claims 1-4, wherein the cardiac failure is cardia failure with a preserved ejection fraction or with a reduced ejection fraction.

6. The method of any one of claims 1-5, wherein the PDE9 inhibitor is administered to the patient at a dose of about 5 mg/kg, about 6 mg/kg, about 7 mg/kg, about 8 mg/kg, or about 9 mg/kg per body weight.

7. The method of any one of claims 1-5, wherein the PDE9 inhibitor is administered to the patient at a dose of about 5 mg/kg or about 8 mg/kg per body weight.

8. A method for treating cardiac fibrosis in a patient in need thereof, the method comprising administering to the patient a therapeutically acceptable dose of a PDE9 inhibitor of Formula (I), or a pharmaceutically acceptable salt or solvate thereof: 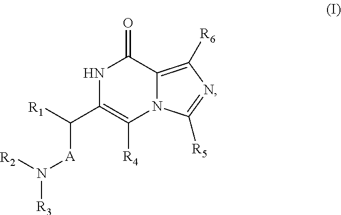
wherein R² is cyclized with either R¹ or R³;
wherein R, R², and R³ are
R¹, when cyclized with R² is 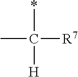
wherein R⁷ is selected from the group consisting of H, —CH₃, —C₂H₅, and C₃H₇;
wherein * denotes the cyclization point; and
R¹, when not cyclized, is selected from the group consisting of H and 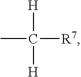
wherein R⁷ is selected from the group consisting of H, —CH₃, —C₂H₅, and C₃H₇;
R² is a compound selected from the group consisting of 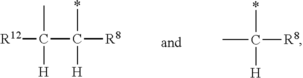
wherein R⁸ and R¹² independently are selected from the group consisting of H, —CH₃, —C₂H₆, and —C₃H₇,
wherein * denotes the cyclization point; and
R³, when cyclized with R² is 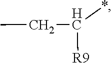
wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C3-C9 heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R³, when not cyclized, is 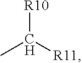
wherein
R¹⁰ is selected from the group consisting of H, —CH₃, and —C₂H₅; and
R¹¹ is selected from the group consisting of C₆-C₁₉ aryl, substituted C₆-C₁₀ aryl, C₃-C₉ heteroaryl, substituted C₃-C₉ heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R⁶ is selected from the group consisting of hydrogen, F, Cl, CN, —CH₃, —C₂H₅, —C₃H₇, and —CF₃; and
A is absent or —CH₂.

9. The method of claim 8, wherein the treating of cardiac fibrosis further comprise decreasing accumulation of fibronectin and/or collagen type I and II.

10. A method of reducing myocardial inflammation in a patient in need thereof, the method comprising administering to the patient a therapeutically acceptable dose of a PDE9 inhibitor of Formula (I), or a pharmaceutically acceptable salt or solvate thereof: 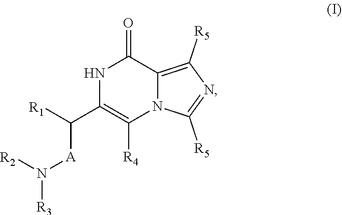
wherein R² is cyclized with either R¹ or R³;
wherein R¹, R², and R³ are
R¹, when cyclized with R² is 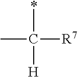
wherein R⁷ is selected from the group consisting of H, —CH₃, —C₂H₅, and C₃H₇;
wherein * denotes the cyclization point; and
R¹, when not cyclized, is selected from the group consisting of H and 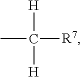
wherein R⁷ is selected from the group consisting of H, —CH₃, —C₂H₅, and C₃H₇;
R² is a compound selected from the group consisting of 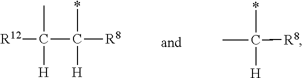
wherein R⁸ and R¹² independently are selected from the group consisting of H, —CH₃, —C₂H₆, and —C₃H₇,
wherein * denotes the cyclization point; and
R³, when cyclized with R² is 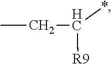
wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R³, when not cyclized, is 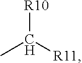
wherein
R¹⁰ is selected from the group consisting of H, —CH₃, and —C₂H₅; and
R¹¹ is selected from the group consisting of C₆-C₁₉ aryl, substituted C₆-C₁₀ aryl, C₃-C₉ heteroaryl, substituted C3-C9 heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C₃-C₆ heterocyclyl, substituted C₃-C₆ heterocyclyl, C₃-C₆ cycloalkyl, and substituted C₃-C₆ cycloalkyl;
R⁶ is selected from the group consisting of hydrogen, F, Cl, CN, —CH₃, —C₂H₅, —C₃H₇, and —CF₃; and
A is absent or —CH₂.

11. A method of decreasing ANP (atrial natriuretic peptide) and/or BNP (B-type natriuretic peptide) in a patient in need thereof, the method comprising administering to the patient a therapeutically acceptable dose of a PDE9 inhibitor of Formula (I), or a pharmaceutically acceptable salt or solvate thereof: 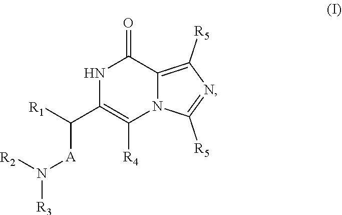
wherein R² is cyclized with either R¹ or R³;
wherein R¹, R², and R³ are
R¹, when cyclized with R² is 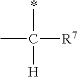
wherein R⁷ is selected from the group consisting of H, —CH₃, —C₂H₅, and C₃H₇;
wherein * denotes the cyclization point; and
R¹, when not cyclized, is selected from the group consisting of H and 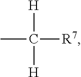
wherein R⁷ is selected from the group consisting of H, —CH₃, —C₂H₅, and C₃H₇;
R² is a compound selected from the group consisting of 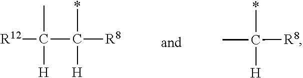
wherein R⁸ and R¹² independently are selected from the group consisting of H, —CH₃, —C₂H₆, and —C₃H₇,
wherein * denotes the cyclization point; and
R³, when cyclized with R² is 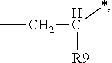
wherein * denotes the cyclization point, and
wherein R⁹ is selected from the group consisting of H, C₁-C₆ alkyl, substituted C₁-C₆ alkyl, branched C₃-C₆ alkyl, C₃-C₆ cycloalkyl, substituted C₃-C₆ cycloalkyl, C₆-C₁₀ aryl, substituted C₆-C₁₀ aryl, C₃-C₉ heteroaryl, substituted C₃-C₉ heteroaryl, C₁-C₆ alkoxy, substituted C₁-C₆ alkoxy, branched C₃-C₆ alkoxy, C₃-C₆ cycloalkoxy, substituted C₃-C₆ cycloalkoxy, C₆-C₁₀ aryloxy, substituted C₆-C₁₀ aryloxy, C₃-C₉ heteroaryloxy, substituted C₃-C₉ heteroaryloxy; and
R³, when not cyclized, is 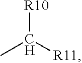
wherein
R¹⁰ is selected from the group consisting of H, —CH₃, and —C₂H₅; and
R¹¹ is selected from the group consisting of C₆-C₁₉ aryl, substituted C₆-C₁₀ aryl, C₃-C₉ heteroaryl, substituted C₃-C₉ heteroaryl;
R⁴ is selected from the group consisting of hydrogen, —CH₃, —C₂H₅, —C₃H₇, —CF₃, —CN, F and Cl;
R⁵ is selected from the group consisting of C₆-C₁₀ aryl, substituted C₆-C₁₀ aryl, C₃-C₉ heteroaryl, substituted C₃-C₉ heteroaryl, C3-C6 heterocyclyl, substituted C3-C6 heterocyclyl, C₃-C₆ cycloalkyl, and substituted C₃-C₆ cycloalkyl;
R⁶ is selected from the group consisting of hydrogen, F, Cl, CN, —CH₃, —C₂H₅, —C₃H₇, and —CF₃; and
A is absent or —CH₂.

12. The method of claim 11, wherein ANP is decrease by about 5%, 10%, 20%, 30%, 40%, or 50%, or more compared to pretreatment levels.

13. The method of claim 11, wherein BNP is decrease by about 5%, 10%, 20%, 30%, 40%, or 50%, or more compared to pretreatment levels.

14. The method of any one of claim 8-13, wherein the PDE9 inhibitor of Formula (I) is 6-[(3S,4S)-4-methyl-1-(pyrimidin-2-ylmethyl)pyrrolidin-3-yl]-3-tetrahydropyran-4-yl-7H-imidazo[1,5-a]pyrazin-8-one (Compound 1), or a pharmaceutically acceptable salt or solvate thereof.

15. The method of any one of claims 8-13, wherein the PDE9 inhibitor is administered to the patient at a dose of between about 10 mg/kg to about 500 mg/kg per body weight.

16. The method of any one of claims 8-13, wherein the PDE9 inhibitor is administered to the patient at a dose of about 50 mg/kg, about 100 mg/kg, about 150 mg/kg, about 200 mg/kg, or about 250 mg/kg per body weight.

17. The method of any one of claims 8-13, wherein the PDE9 inhibitor is administered to the patient at a dose of about 60 mg/kg or about 100 mg/kg per body weight.

18. The method of any one of claims 8-13, wherein the PDE9 inhibitor is administered to the patient at at about 100 mg to about 800 mg per dose.

19. The method of any one of claims 8-13 wherein the PDE9 inhibitor is administered to the patient at about 100 mg, 200 mg, 300 mg, 400 mg, 500 mg, 600 mg, 700 mg or 800 mg per dose.

20. The method of any one of claims 1-19, wherein the PDE9 inhibitor is administered to the patient at QD, BID, or TID.

21. The method of any one of claims 1-20, wherein the PDE9 is administered with at least one additional therapeutic agent.

22. The method of claim 21, wherein the additional therapeutic agent is selected from an angiotensin transferase inhibitor (ACEI), a β-receptor blocker, a mineralocorticoid/aldosterone receptor antagonist (MRA), a diuretic, an angiotensin receptor neprilysin inhibitor (ARNI), a neprilysin inhibitor (NEPI), an angiotensin II receptor blocker (ARB), a vasodilator, and a hydralazine (HYD) or isosorbide dinitrate (SND), or a combination thereof.

23. The method of claim 21 or 22, wherein the addtional therapeutic agent is selected from hydroxy urea (HU), captopril, enalapril, lisinopril, trandolapril, bisoprolol, carvedilol, metoprolol succinate, nebivolol, eplerenone, spirolactone, sacubitril, ivabradine, candesartan, valsartan, digoxin, deslanoside, dopamine, dobutamine, dopexamine, milrinone, enoximone, phosphocreatine, cyclohexylethylamine, nitroglycerin, isosorbide dinitrate, sodium nitroprusside, prazosin, ivabradine, candesartan, valsartan, furosemide, bumetanide, torasemide, bendrofluazide, hydrochlorothiazide, metolazone, indapamide, amiloride, and triamterene.

24. The method of claim 22 or 23, wherein the addtional therapeutic agent is angiotensin II.

25. The method of any one of claims 21-23, wherein the PDE9 inhibitor and the a least one additional therapeutic agent are administed concurrently or sequentially.

26. The method of any one of claims 1-25, wherein the PDE9 inhibitor is administerd orally.

27. The method of any one of claims 1-26, whrein the PDE9 inhibitor is administerd daily.

28. The method of any one of claims 1-27, wherein the PDE9 inhibitor is administerd for between 1 to 7 days.

29. The method of any one of claims 1-27, wherein the PDE9 inhibitor is administed for at least 7 days.
